(19) United States
(12) Patent Application Publication
(10) Pub. No.: US 2024/0066155 A1
(43) Pub. Date: Feb. 29, 2024

(54) DUAL MODE RADIOTRACER AND -THERAPEUTICS

(71) Applicants: TECHNISCHE UNIVERSITÄT MÜNCHEN, München (DE); TECHNISCHE UNIVERSITÄT MÜNCHEN - KLINIKUM RECHTS DER ISAR, München (DE)

(72) Inventors: Hans-Jürgen WESTER, Schweitenkirchen (DE); Alexander Josef WURZER, München (DE); Matthias Johannes EIBER, Vaterstetten (DE)

(21) Appl. No.: 18/260,272

(22) PCT Filed: Jan. 4, 2022
(86) PCT No.: PCT/EP2022/050081

§ 371 Date: Jul. 3, 2023

(30) Foreign Application Priority Data
Jan. 4, 2021 (EP) 21150122.6

(51) Int. Cl. A61K 51/04; A61P 35/00

(52) U.S. Cl. CPC A61K 51/0402; A61K 51/0497; A61P 35/00

(57) ABSTRACT

The present invention relates to compounds of the Formula (1) and pharmaceutically acceptable salts thereof, wherein M³⁺ is as defined herein, and their use as cancer diagnostic or imaging agents.

The present invention relates to compounds that bind to prostate-specific membrane antigen (PSMA) comprising a PSMA binding moiety, a linker group comprising a silicon-fluoride acceptor (SI FA) moiety and a chelator moiety, wherein the SI FA moiety comprises a covalent bond between a silicon and a fluorine atom which can be ¹⁸F, and use of the compounds as cancer diagnostic or imaging agents.

### BACKGROUND OF THE INVENTION

### Prostate Cancer

Prostate Cancer (PCa) remained over the last decades the most common malignant disease in men with high incidence for poor survival rates. Due to its overexpression in prostate cancer (Silver et al., Clinical Cancer Research 3, 81-85 (1997)), prostate-specific membrane antigen (PSMA) or glutamate carboxypeptidase II (GCP II) proved its eligibility as excellent target for the development of highly sensitive radiolabelled agents for endoradiotherapy and imaging of PCa (Afshar-Oromieh et al., European journal of nuclear medicine and molecular imaging 42, 197-209 (2015); Benes̆ová et al., Journal of Nuclear Medicine 56, 914-920 (2015); Robu et al., Journal of Nuclear Medicine, jnumed. 116.178939 (2016); Weineisen et al.; Journal of Nuclear Medicine 55, 1083-1083 (2014); Rowe et al., Prostate cancer and prostatic diseases (2016); Maurer et al., Nature Reviews Urology (2016)). Prostate-specific membrane antigen is an extracellular hydrolase whose catalytic center comprises two zinc(II) ions with a bridging hydroxido ligand. It is highly upregulated in metastatic and hormone-refractory prostate carcinomas, but its physiologic expression has also been reported in kidneys, salivary glands, small intestine, brain and, to a low extent, also in healthy prostate tissue. In the intestine, PSMA facilitates absorption of folate by conversion of pteroylpoly-y-glutamate to pteroylglutamate (folate). In the brain, it hydrolyses N-acetyl-L-aspartyl-L-glutamate (NAAG) to N-acetyl-L-aspartate and glutamate.

### Prostate-Specific Membrane Antigen (PSMA)

Prostate-specific membrane antigen (PSMA) is a type II transmembrane glycoprotein that is highly overexpressed on prostate cancer epithelial cells. Despite its name, PSMA is also expressed, to varying degrees, in the neovasculature of a wide variety of nonprostate cancers. Among the most common nonprostate cancers to demonstrate PSMA expression include breast, lung, colorectal, and renal cell carcinoma.

The general necessary structures of PSMA targeting molecules comprise a binding unit that encompasses a zinc-binding group (such as urea (Zhou et al., Nature Reviews Drug Discovery 4, 1015-1026 (2005)), phosphinate or phosphoramidate) connected to a P1′ glutamate moiety, which warrants high affinity and specificity to PSMA and is typically further connected to an effector functionality (Machulkin et al., Journal of drug targeting, 1-15 (2016)). The effector part is more flexible and to some extent tolerant towards structural modifications. The entrance tunnel accommodates two other prominent structural features, which are important for ligand binding. The first one is an arginine patch, a positively charged area at the wall of the entrance funnel and the mechanistic explanation for the preference of negatively charged functionalities at the P1 position of PSMA. This appears to be the reason for the preferable incorporation of negative charged residues within the ligand-scaffold. An in-depth analysis about the effect of positive charges on PSMA ligands has been, to our knowledge, so far not conducted. Upon binding, the concerted repositioning of the arginine side chains can lead to the opening of an S1 hydrophobic accessory pocket, the second important structure that has been shown to accommodate an iodo-benzyl group of several urea based inhibitors, thus contributing to their high affinity for PSMA (Barinka et al., Journal of medicinal chemistry 51, 7737-7743 (2008)).

Zhang et al. discovered a remote binding site of PSMA, which can be employed for bidentate binding mode (Zhang et al., Journal of the American Chemical Society 132, 12711-12716 (2010)). The so called arene-binding site is a simple structural motif shaped by the side chains of Arg463, Arg511 and Trp541, and is part of the GCPII entrance lid. The engagement of the arene binding site by a distal inhibitor moiety can result in a substantial increase in the inhibitor affinity for PSMA due to avidity effects. PSMA I&T was developed with the intention to interact this way with PSMA, albeit no crystal structure analysis of binding mode is available. A necessary feature according to Zhang et al. is a linker unit (Suberic acid in the case of PSMA I&T) which facilitates an open conformation of the entrance lid of GCPII and thereby enabling the accessibility of the arene-binding site. It was further shown that the structural composition of the linker has a significant impact on the tumor-targeting and biologic activity as well as on imaging contrast and pharmacokinetics (Liu et al., Bioorganic & medicinal chemistry letters 21, 7013-7016 (2011)), properties which are crucial for both high imaging quality and efficient targeted endoradiotherapy.

Two categories of PSMA-targeting inhibitors are currently used in clinical settings. On the one side there are tracers with chelating units for radionuclide complexation such as PSMA I&T or related compounds (Kiess et al., The quarterly journal of nuclear medicine and molecular imaging 59, 241 (2015)). On the other side there are small molecules, comprising a targeting unit and effector molecules.

The most often used agents for selective PSMA imaging are PSMA HBED-CC (Eder et al., Bioconjugate chemistry 23, 688-697 (2012)), PSMA-617 (Bene§ova et al., Journal of Nuclear Medicine 56, 914-920 (2015)) and PSMA I&T (Weineisen et al.; Journal of Nuclear Medicine 55, 1083-1083 (2014)), which are predominantly labelled with ⁶⁸Ga (88.9%, β^{+, E}_{β+, max}=1.89 MeV, t_{1/2}=68 min). Among these ⁶⁸Ga-PSMA-HBED-CC (also known as ⁶⁸Ga-PSMA-11), is so far considered as the golden standard for PET imaging of PCa.

### ¹⁸F Labelling

Recently, several groups have focused on the development of novel ¹⁸F-labelled urea-based inhibitors for PCa diagnosis. In contrast to the radiometal ⁶⁸Ga, which can be obtained from commercially distributed ⁶⁸Ge/⁶⁸Ga radionuclide generators (⁶⁸Ge; t_{1/2}=270.8 d), the radioisotope ¹⁸F-fluorine (96.7% β⁺, E_{β+, max}=634 keV) requires an on-site cyclotron for its production. Despite this limitation, ¹⁸F offers due to its longer half-live (t_{1/2}=109.8 min) and its lower positron energy, significant advantages in terms of routine-handling and image quality. Additionally, there is the possibility for largescale production in a cyclotron, which would be beneficial for a higher patient throughput and reduction of production costs. The ¹⁸F-labelled urea-based PSMA inhibitor ¹⁸F-DCFPyl demonstrated promising results in the detection of primary and metastatic PCa (Rowe et al., Molecular Imaging and Biology, 1-9 (2016)) and superiority to ⁶⁸Ga-PSMA-HBED-CC in a comparative study (Dietlein et al., Molecular Imaging and Biology 17, 575-584 (2015)). Based on the structure of PSMA-617, the ¹⁸F-labelled analogue PSMA-1007 was recently developed, which showed comparable tumor-to-organ ratios (Cardinale et al., Journal of nuclear medicine: official publication, Society of Nuclear Medicine 58, 425-431 (2017); Giesel et al., European journal of nuclear medicine and molecular imaging 43, 1929-1930 (2016)). A comparative study with ⁶⁸Ga-PSMA-HBED-CC revealed similar diagnostic accuracy of both tracers and a reduced urinary clearance of ¹⁸F-PSMA-1007, enabling a better assessment of the prostate (Giesel et al., European journal of nuclear medicine and molecular imaging 44, 678-688 (2017)).

An attractive approach for introducing ¹⁸F labels is the use of silicon fluoride acceptors (SIFA). Silicon fluoride acceptors are described, for example, in Lindner et al., Bioconjugate Chemistry 25, 738-749 (2014). In order to preserve the silicon-fluoride bond, the use of silicon fluoride acceptors introduces the necessity of sterically demanding groups around the silicone atom. This in turn renders silicon fluoride acceptors highly hydrophobic. In terms of binding to the target molecule, in particular to the target molecule which is PSMA, the hydrophobic moiety provided by the silicone fluoride acceptor may be exploited for the purpose of establishing interactions of the radio-diagnostic or -therapeutic compound with the hydrophobic pocket described in Zhang et al., Journal of the American Chemical Society 132, 12711-12716 (2010). Yet, prior to binding, the higher degree of lipophilicity introduced into the molecule poses a severe problem with respect to the development of radiopharmaceuticals with suitable in vivo biodistribution, i.e. low unspecific binding in non-target tissue.

### Failure to Solve the Hydrophobicity Problem

Despite many attempts, the hydrophobicity problem caused by silicon fluoride acceptors has not been satisfactorily solved in the prior art.

To explain further, Schirrmacher E. et al. (Bioconjugate Chem. 2007, 18, 2085-2089) synthesized different ¹⁸F-labelled peptides using the highly effective labelling synthon p-(di-tert-butylfluorosilyl) benzaldehyde ([¹⁸F]SIFA-A), which is one example of a silicon fluoride acceptor. The SIFA technique resulted in an unexpectedly efficient isotopic 19F-¹⁸F exchange and yielded the ¹⁸F-synthon in almost quantitative yields in high specific activities between 225 and 680 GBq/μmol (6081-18 378 Ci/mmol) without applying HPLC purification. [¹⁸F]SIFA-benzaldehyde was finally used to label the N-terminal amino-oxy (N-AO) derivatized peptides AO-Tyr3 -octreotate (AO-TATE), cyclo(fK(AO-N)RGD) and N-AO-PEG₂-[D-Tyr-Gln-Trp-Ala-Val-Ala-His-Thi-Nle-NH₂](AO-BZH3, a bombesin derivative) in high radiochemical yields. Nevertheless, the labelled peptides are highly lipophilic (as can be taken from the HPLC retention times using the conditions described in this paper) and thus are unsuitable for further evaluation in animal models or humans.

In Wängler C. et al. (Bioconjugate Chem., 2009, 20 (2), pp 317-321), the first SIFA-based Kit-like radio-fluorination of a protein (rat serum albumin, RSA) has been described. As a labelling agent, 4-(di-tert-butyl[¹⁸F]fluorosilyl)benzenethiol (Si[¹⁸F]FA-SH) was produced by simple isotopic exchange in 40-60% radiochemical yield (RCY) and coupled the product directly to maleimide derivatized serum albumin in an overall RCY of 12% within 20-30 min. The technically simple labelling procedure does not require any elaborated purification procedures and is a straightforward example of a successful application of Si—¹⁸F chemistry for in vivo imaging with PET. The time-activity cureves and μPET images of mice showed that most of the activity was localized in the liver, thus demonstrating that the labelling agent is too lipophilic and directs the in vivo probe to hepatobiliary excretion and extensive hepatic metabolism.

Wängler C. et al. (see Bioconjug Chem. 2010 Dec. 15; 21(12): 2289-96) subsequently tried to overcome the major drawback of the SIFA technology, the high lipophilicity of the resulting radiopharmaceuticals, by synthesizing and evaluating new SIFA-octreotate analogues (SIFA-Tyr3-octreotate, SIFA-Asn(AcNH-β-Glc)-Tyr3-octreotate and SIFA-Asn(AcNH-β-Glc)-PEG-Tyr3-octreotate). In these compounds, hydrophilic linkers and pharmacokinetic modifiers were introduced between the peptide and the SIFA-moiety, i.e. a carbohydrate and a PEG linker plus a carbohydrate. As a measure of lipophilicity of the conjugates, the log P(ow) was determined and found to be 0.96 for SIFA-Asn(AcNH-β-Glc)-PEG-Tyr³-octreotate and 1.23 for SIFA-Asn(AcNH-β-Glc)-Tyr³-octreotate. These results show that the high lipophilicity of the SIFA moiety can only be marginally compensated by applying hydrophilic moieties. A first imaging study demonstrated excessive hepatic clearance /liver uptake and thus has never been transferred into a first human study.

Bernard-Gauthier et al. (Biomed Res Int. 2014; 2014: 454503) reviews a great plethora of different SIFA species that have been reported in the literature ranging from small prosthetic groups and other compounds of low molecular weight to labelled peptides and most recently affibody molecules. Based on these data the problem of lipophilicity of SIFA-based prosthetric groups has not been solved sofar; i.e. a methodology that reduces the overall lipophilicity of a SIFA conjugated peptide to a log D lower than approx. −2,0 has not been described.

In Lindner S. et al. (Bioconjug Chem. 2014 Apr. 16; 25(4): 738-49) it is described that PEGylated bombesin (PESIN) derivatives as specific GRP receptor ligands and RGD (one-letter codes for arginine-glycine-aspartic acid) peptides as specific avp3 binders were synthesized and tagged with a silicon-fluoride-acceptor (SIFA) moiety. To compensate the high lipophilicity of the SIFA moiety various hydrophilic structure modifications were introduced leading to reduced logD values. SIFA-Asn(AcNH-β-Glc)-PESIN, SIFA-Ser(β-Lac)-PESIN, SI FA-Cya-PESIN, SIFA-LysMe3-PESIN, SIFA-γ-carboxy-d-Glu-PESIN, SI FA-Cya2-PESIN, SIFA-LysMe3-γ-carboxy-d-Glu-PESIN, SIFA-(γ-carboxy-d-Glu)2-PESIN, SIFA-RGD, SIFA-γ-carboxy-d-Glu-RGD, SIFA-(γ-carboxy-d-Glu)2-RGD, SI FA-LysMe3-γ-carboxy-d-Glu-RGD. All of these peptides—already improved and derivatized with the aim to reduce the lipophilicity—showed a logD value in the range between +2 and −1.22.

In Niedermoser S. et al. (J Nucl Med. 2015 Jul; 56(7): 1100-5), newly developed ¹⁸F-SIFA- and ¹⁸F-SIFAlin-(SIFA=silicon-fluoride-acceptor) modified TATE derivatives were compared with the current clinical gold standard ⁶⁸Ga-DOTATATE for high-quality imaging of somatostatin receptor-bearing tumors. For this purpose, ¹⁸F-SIFA-TATE and two quite complex analogues, ¹⁸F-SIFA-Glc-PEG1-TATE, ¹⁸F-SIFAlin-Glc-Asp2-PEG1-TATE were developed. None of the agents showed a logD<-1.5.

In view of the above, the technical problem underlying the present invention can be seen in providing radio-diagnostics and radio-therapeutics which contain a silicone fluoride acceptor and which are, at the same time, characterized by favourable in-vivo properties.

WO2019/020831 and WO2020/157184 disclose ligand-SIFA-chelator conjugates.

In the present invention a proof-of-principle has been established using specific conjugates which bind with high affinity to prostate-specific membrane antigen (PSMA) as target. Accordingly, a further technical problem underlying the present invention can be seen in providing improved radio-therapeutics and—diagnostics for the medical indication which is cancer, preferably prostate cancer.

### THE INVENTION

The present invention relates to compounds of the Formula (1):

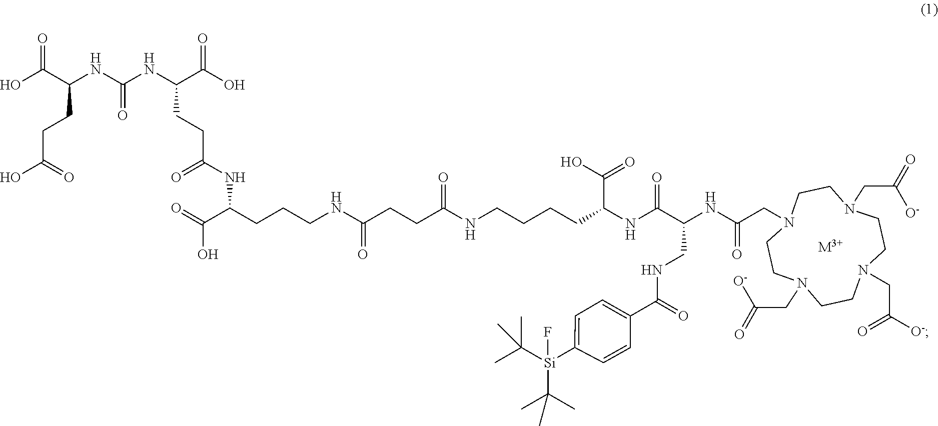

and pharmaceutically acceptable salts thereof, wherein M³⁺ is a chelated radioactive or non-radioactive metal cation.

Also provided is a pharmaceutical or diagnostic composition comprising a compound of the invention. The compound of the invention may be for use as a cancer diagnostic or imaging agent. Accordingly also provided is a method of imaging and/or diagnosing cancer comprising administering a compound of the invention or a composition comprising a compound of the invention. The compounds or compositions of the invention may be for use in the treatment of cancer. The compounds or compositions of the invention may be for use in the diagnosis, imaging or prevention of neoangiogenesis/angiogenesis. The compounds or compositions of the invention may be for use as a cancer diagnostic or imaging agent or for use in the treatment of cancer. The compounds or compositions of the invention may be for use as a cancer diagnostic or imaging agent or for use in the treatment of cancer wherein the cancer is prostate, breast, lung, colorectal or renal cell carcinoma.

### DETAILED DESCRIPTION OF THE INVENTION

The present invention relates to compounds of the Formula (1):

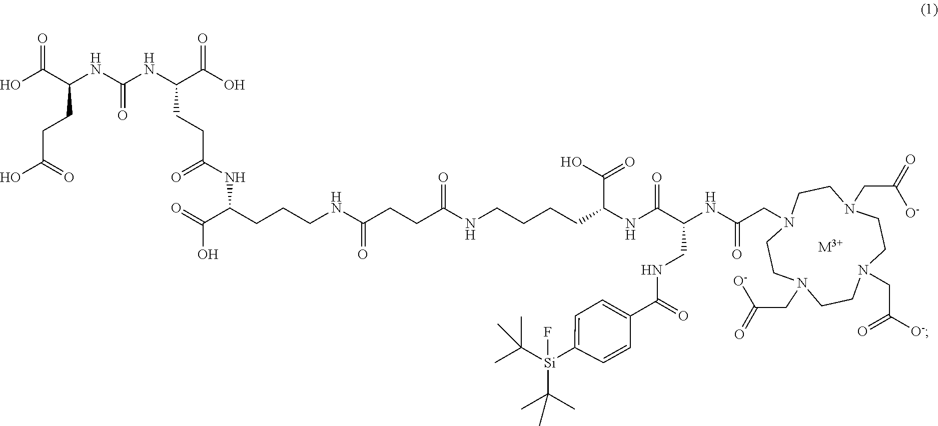

and pharmaceutically acceptable salts thereof, wherein M³⁺ is a chelated radioactive or non-radioactive metal cation.

Also included are pharmaceutical or diagnostic compositions comprising one or more compounds of the invention.

In the compounds of Formula (1), F may be any isotope of fluorine. In the compounds of Formula (1), F may be ¹⁸F or ¹⁹F. In the compounds of Formula (1), F may be ¹⁸F. In the compounds of Formula (1), F may be ¹⁹F. In compositions comprising compounds of the Formula (1) any combination of fluorine isotopes may be present. In compositions comprising compounds of the Formula (1) any combination of ¹⁸F and ¹⁹F may be present. In particular, compounds and compositions of the invention include compounds of Formula (1) where F is ¹⁸F or ¹⁹F. Compounds of the invention for use as diagnostic or imaging agents include compounds of Formula (1) where F is ¹⁸F. Compounds of the invention for use as therapeutic agents include compounds of Formula (1) where F is ¹⁹F.

In the compounds herein, M³⁺ can be selected from the cations of Sc, Cu, Ga, Y, In, Tb, Ho, Lu, Re, Pb, Bi, Ac, Er and Th. M³⁺ can be selected from Sc³⁺, Cu³⁺, Ga³⁺, Y³⁺, In³⁺, Tb³⁺, Ho³⁺, Lu³⁺, Re³⁺, Pb³⁺, Bi³⁺, Ac³⁺, Er³⁺ and Th³⁺. M³⁺ can be Ga³⁺ or Lu³⁺. M³⁺ can be Ga³⁺. M³⁺ can be ⁶⁸Ga³⁺. M³⁺ can be Lu³⁺. M³⁺ can be ¹⁷⁷Lu³⁺. M³⁺ can be Ac³⁺. M³⁺ can be ²²⁵Ac³⁺.

Particular compounds include:

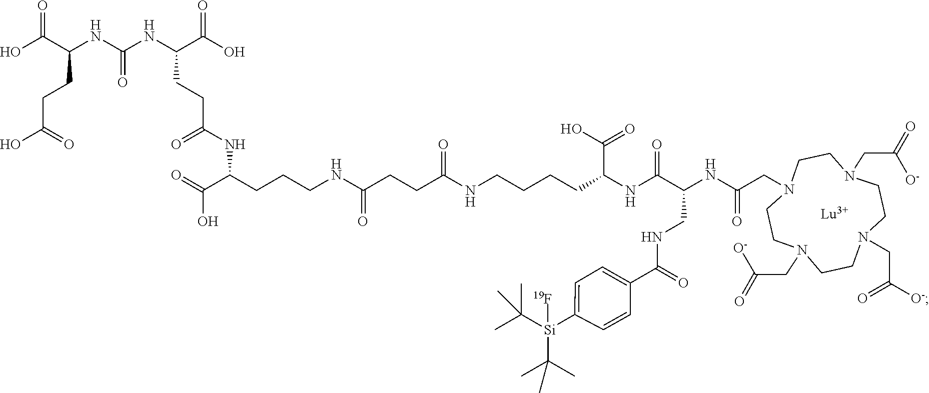

and pharmaceutically acceptable salts thereof.

Particular compounds include:

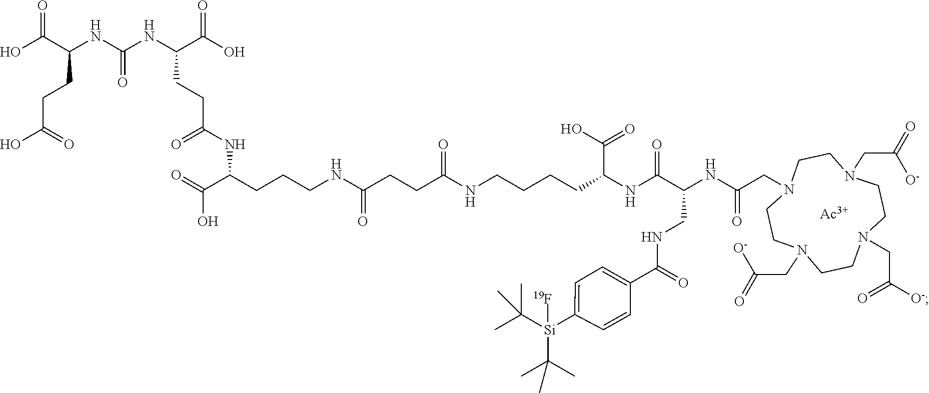

and pharmaceutically acceptable salts thereof.

Particular compounds include:

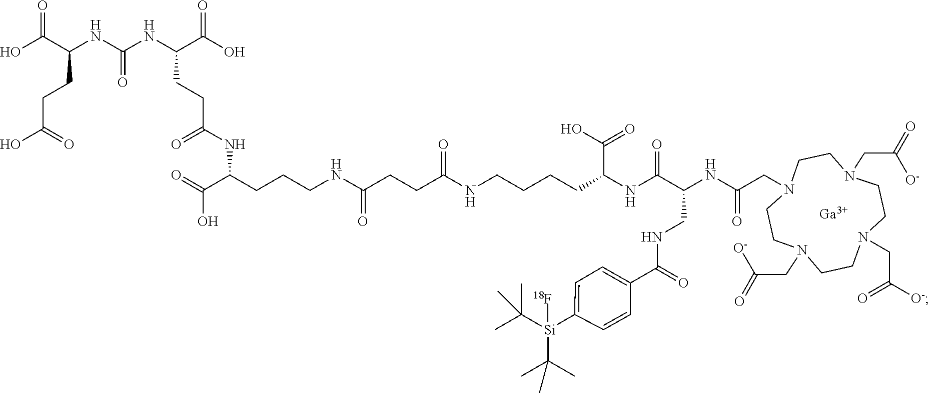

and pharmaceutically acceptable salts thereof.

The compounds or compositions of the invention may be for use as a cancer diagnostic or imaging agent.

The compounds or compositions of the invention may be for use in the treatment of cancer.

The compounds or compositions of the invention may be for use in the treatment of cancer wherein the cancer is prostate, breast, lung, colorectal or renal cell carcinoma.

The compounds or compositions of the invention may be for use as a cancer diagnostic or imaging agent or for use in the treatment of cancer wherein the cancer is prostate, breast, lung, colorectal or renal cell carcinoma.

The compounds or compositions of the invention may be for use in the diagnosis, imaging or prevention of neoangiogenesis/angiogenesis.

Also provided are methods of imaging and/or diagnosing cancer comprising administering a compound or composition of the invention to a patient in need thereof.

Provided are the compounds:

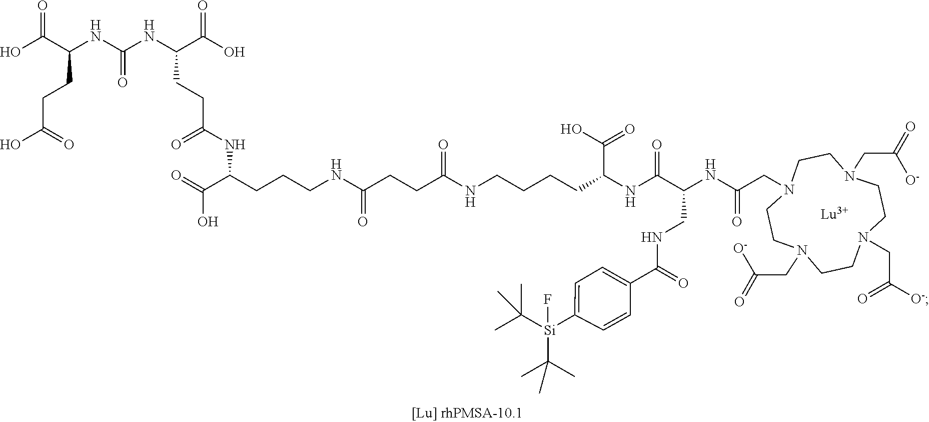

and pharmaceutically acceptable salts thereof.

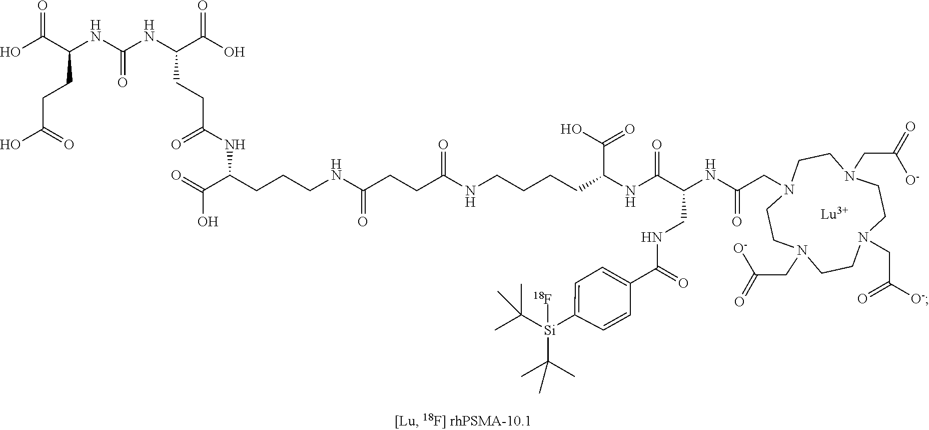

and pharmaceutically acceptable salts thereof.

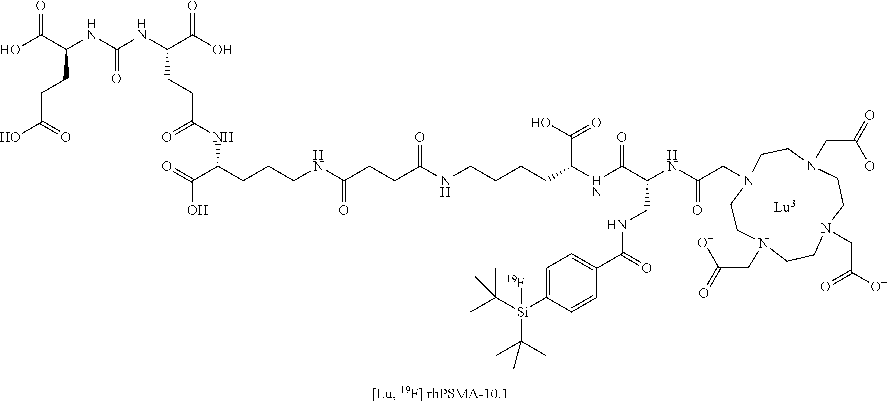

and pharmaceutically acceptable salts thereof.

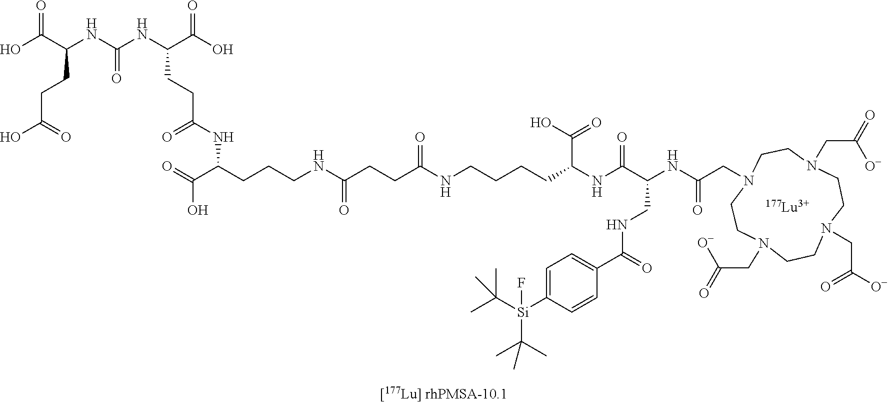

and pharmaceutically acceptable salts thereof.

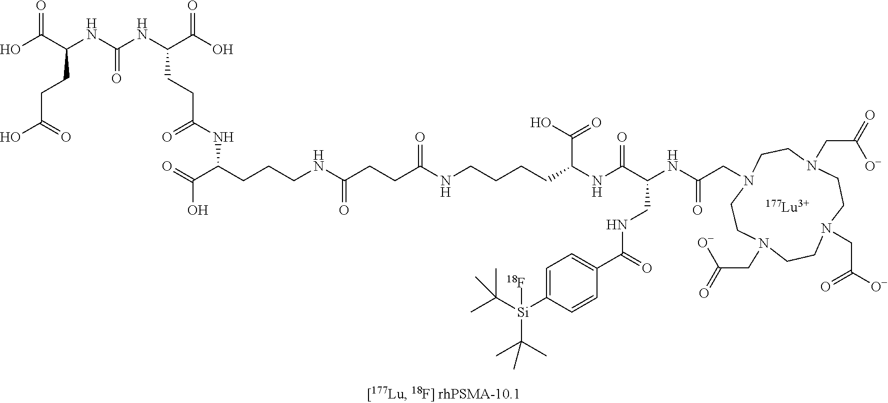

and pharmaceutically acceptable salts thereof.

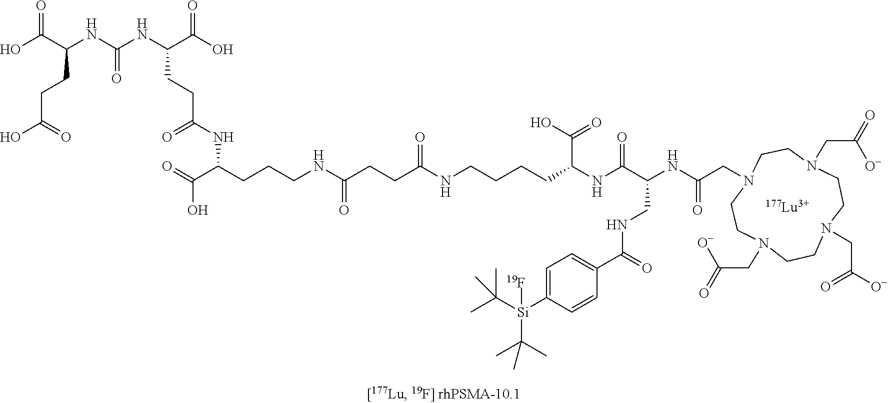

and pharmaceutically acceptable salts thereof.

The Lu may alternatively be Ac, for example ²²⁵AC.

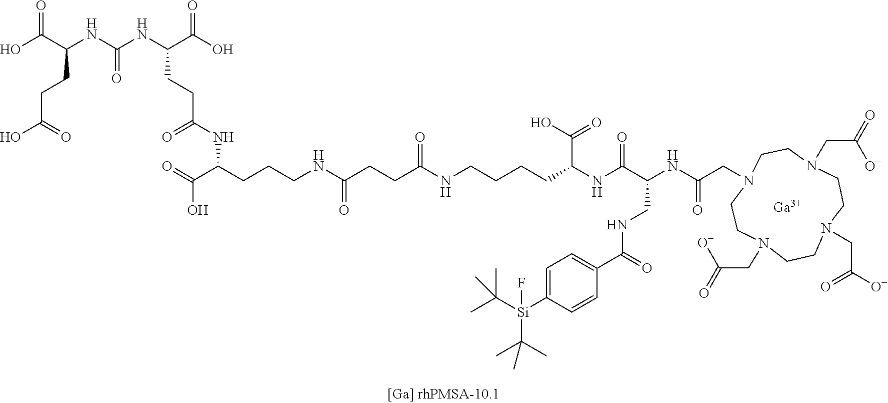

and pharmaceutically acceptable salts thereof.

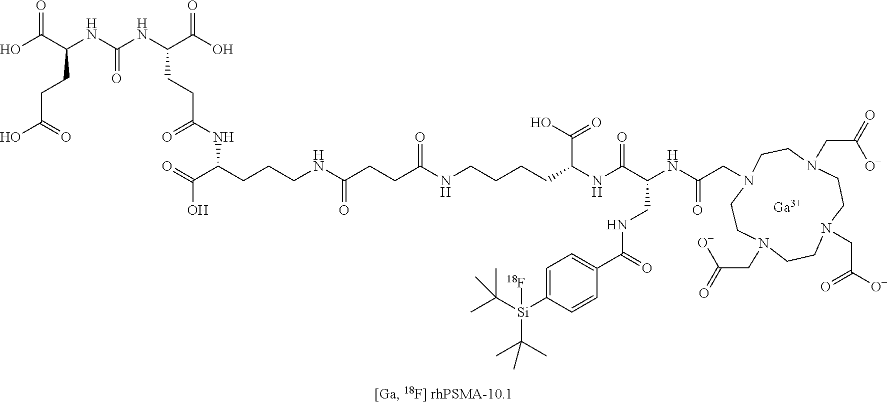

and pharmaceutically acceptable salts thereof.

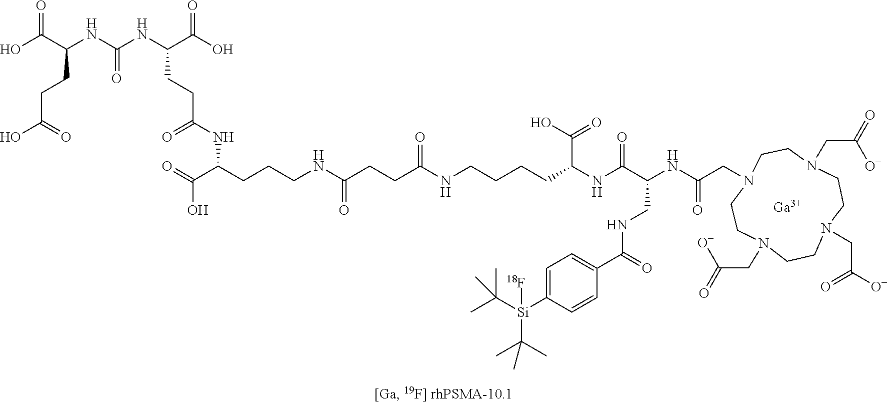

and pharmaceutically acceptable salts thereof.

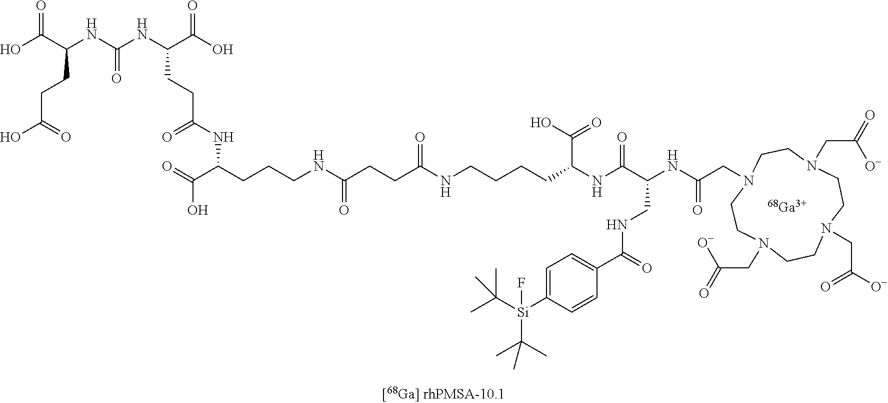

and pharmaceutically acceptable salts thereof.

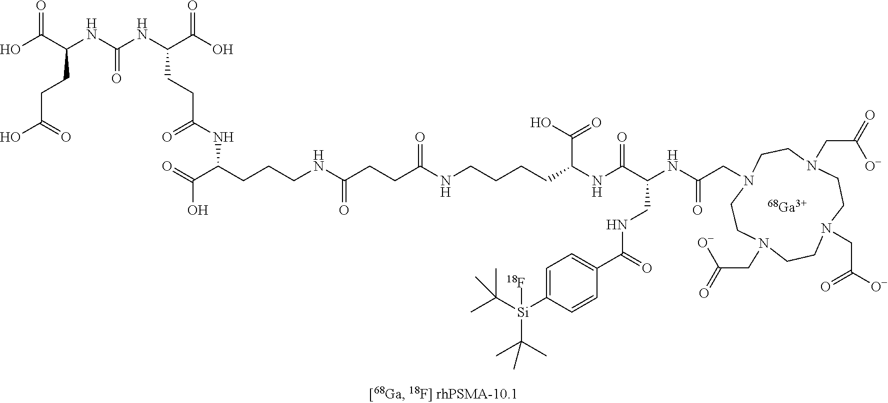

and pharmaceutically acceptable salts thereof.

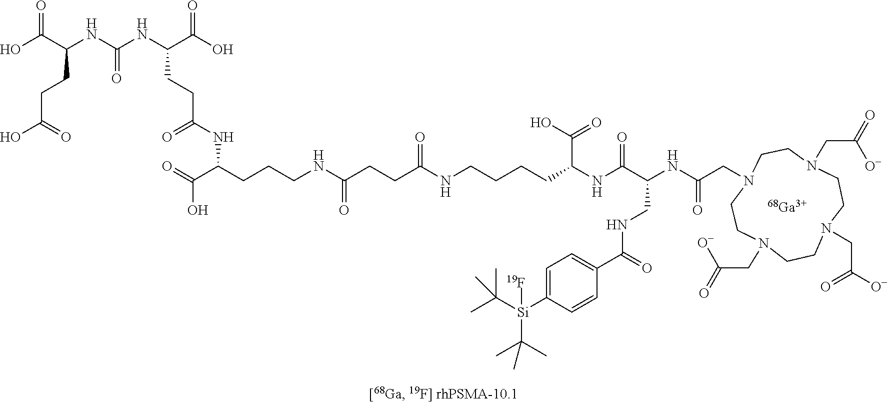

and pharmaceutically acceptable salts thereof.

Also disclosed are the compounds:

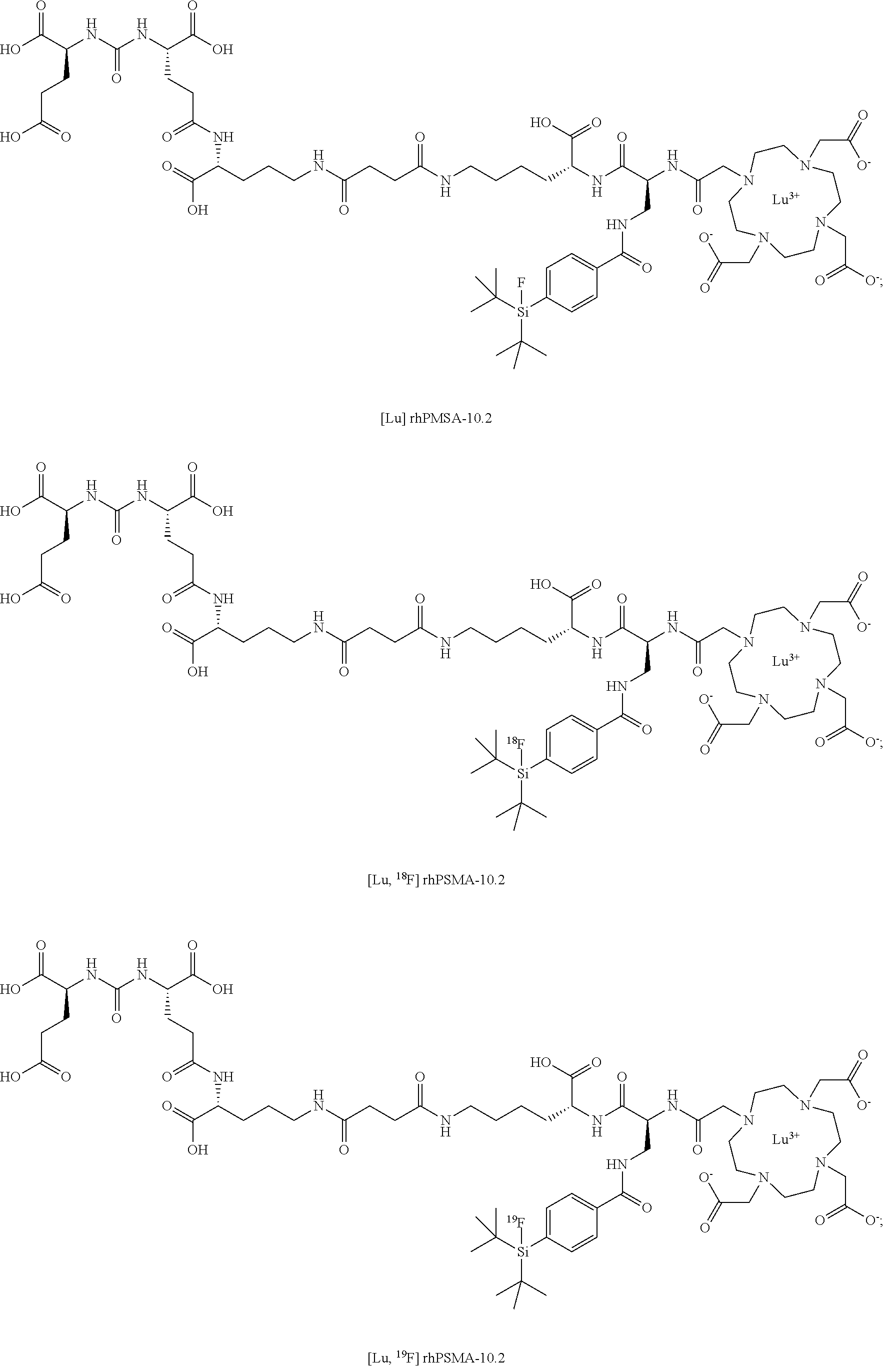

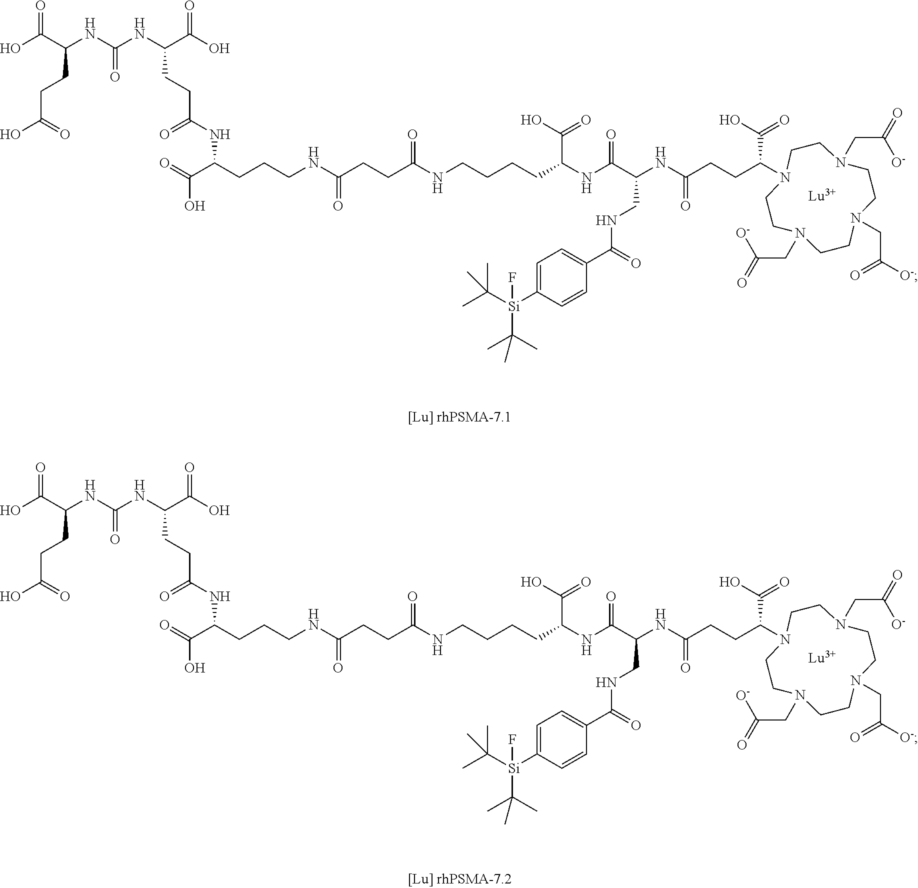

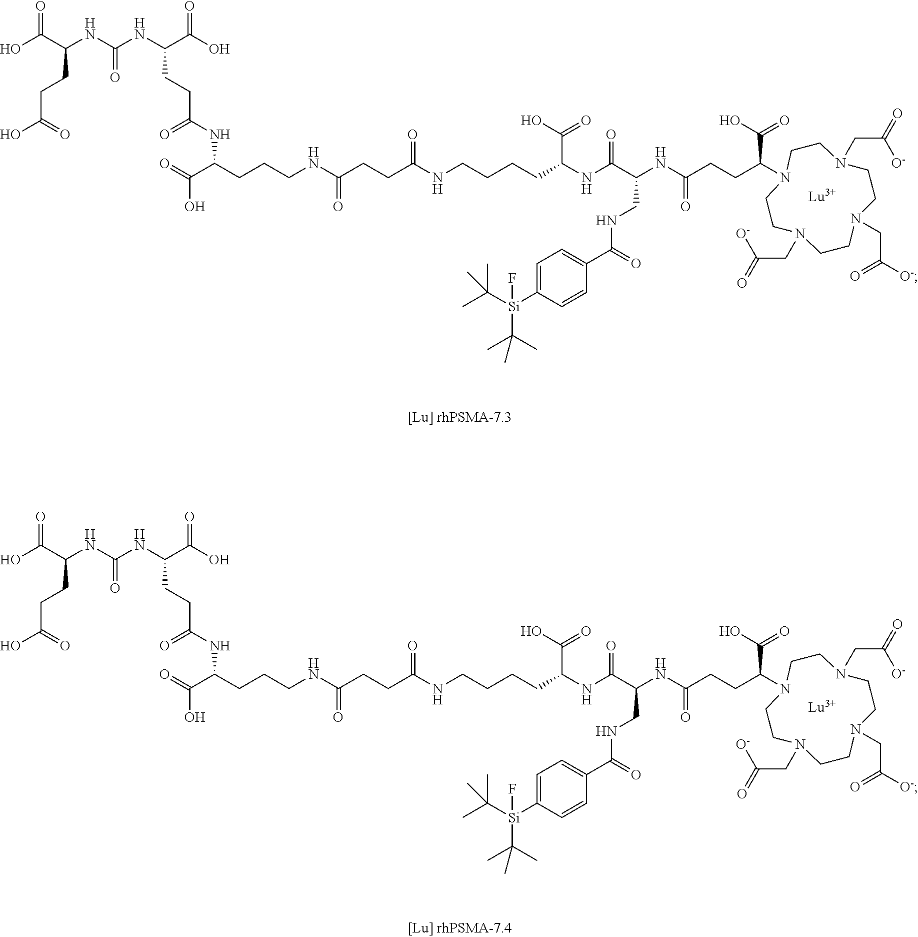

The compounds provided herein comprise three separate moieties. The three separate moieties are a PSMA binding moiety, a linker group comprising a silicon-fluoride acceptor (SIFA) moiety and a chelator moiety, wherein the SIFA moiety comprises a covalent bond between a silicon and a fluorine atom which can be ¹⁸F or ¹⁹F.

For diagnostic imaging, the fluorine atom on the SIFA moiety may be ¹⁸F. The ¹⁸F can be introduced by isotopic exchange with ¹⁹F.

The compounds of the invention require a hydrophilic chelator moiety in addition to the PSMA binding moiety. The hydrophilic chelator moiety is required to reduce the hydrophobic nature of the compounds caused by the presence of the SI FA moiety. A key aspect of the invention is the combination, within a single molecule, of a silicon fluoride acceptor and a chelator moiety or a chelate. These two structural elements, SIFA and the chelator, exhibit a spatial proximity.

The compounds of the invention may be radioactively labelled at the SIFA moiety. Also included are molecules which are not radiolabelled at the SI FA moiety.

The present inventors surprisingly discovered that placement of the silicone fluoride acceptor in the neighbourhood of a hydrophilic chelator such as DOTAGA or DOTA, shields or compensates efficiently the lipophilicity of the SIFA moiety to an extent which shifts the overall hydrophobicity of compound in a range which renders the compound suitable for in-vivo administration.

A further advantage of the compounds of the present invention is their surprisingly low accumulation in the kidneys of mice when compared to other PSMA targeted radiopharmaceuticals, such as PSMA I&T. Without wishing to be bound by a particular theory, it seems to be the combination of the structural element SI FA with a chelator which provides for the unexpected reduction of accumulation in the kidneys.

In terms of lipophilicity/hydrophilicity, the logP value (sometimes also referred to as logD value) is an art-established measure.

The term “lipophilicity” relates to the strength of being dissolved in, or be absorbed in lipid solutions, or being adsorbed at a lipid-like surface or matrix. It denotes a preference for lipids (literal meaning) or for organic or apolar liquids or for liquids, solutions or surfaces with a small dipole moment as compared to water. The term “hydrophobicity” is used with equivalent meaning herein. The adjectives lipophilic and hydrophobic are used with corresponding meaning to the substantives described above.

The mass flux of a molecule at the interface of two immiscible or substantially immiscible solvents is governed by its lipophilicity. The more lipophilic a molecule is, the more soluble it is in the lipophilic organic phase. The partition coefficient of a molecule that is observed between water and n-octanol has been adopted as the standard measure of lipophilicity. The partition coefficient P of a species A is defined as the ratio P=[A]_{n-octanol}/[A]_{water}. A figure commonly reported is the logP value, which is the logarithm of the partition coefficient. In case a molecule is ionizable, a plurality of distinct microspecies (ionized and not ionized forms of the molecule) will in principle be present in both phases. The quantity describing the overall lipophilicity of an ionizable species is the distribution coefficient D, defined as the ratio D=[sum of the concentrations of all microspecies]_{n-octanol}/[sum of the concentrations of all microspecies]_{water}. Analogous to logP, frequently the logarithm of the distribution coefficient, logD, is reported. Often, a buffer system, such as phosphate buffered saline is used as alternative to water in the above described determination of logP.

If the lipophilic character of a substituent on a first molecule is to be assessed and/or to be determined quantitatively, one may assess a second molecule corresponding to that substituent, wherein said second molecule is obtained, for example, by breaking the bond connecting said substituent to the remainder of the first molecule and connecting (the) free valence(s) obtained thereby to hydrogen(s).

Alternatively, the contribution of the substituent to the logP of a molecule may be determined. The contribution π_{X}x of a substituent X to the logP of a molecule R—X is defined as π_{X}x=logP_{R—X}−logP_{R—H}, wherein R—H is the unsubstituted parent compound.

Values of P and D greater than one as well as logP, logD and π_{X}x values greater than zero indicate lipophilic/hydrophobic character, whereas values of P and D smaller than one as well as logP, logD and π_{X}x values smaller than zero indicate hydrophilic character of the respective molecules or substituents.

The above described parameters characterizing the lipophilicity of the lipophilic group or the entire molecule according to the invention can be determined by experimental means and/or predicted by computational methods known in the art (see for example Sangster, Octanol-water Partition Coefficients: fundamentals and physical chemistry, John Wiley & Sons, Chichester. (1997)).

The logP value of compounds of the invention may be between −5 and −1.5. It is particularly preferred that the logP value is between −3.5 and −2.0.

The compounds are preferably high affinity PSMA ligands with preferable affinity, expressed as IC₅₀, being below 50 nM, below 20 nM or below 5 nM.

In order to be used in PET imaging, the compounds require a positron emitting atom. The compounds include ¹⁸F for medical use.

Also provided is a pharmaceutical imaging composition comprising or consisting of one or more compounds of the invention as disclosed herein.

Also provided is a diagnostic composition comprising or consisting of one or more compounds of the invention as disclosed herein.

The pharmaceutical composition may further comprise pharmaceutically acceptable carriers, excipients and/or diluents. Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well-known conventional methods. These pharmaceutical compositions can be administered to the subject at a suitable dose. Administration of the suitable compositions may be effected in different ways, e.g., by intravenous, intraperitoneal, subcutaneous, intramuscular, topical, intradermal, intranasal or intrabronchial administration. It is particularly preferred that said administration is carried out by injection and/or delivery, e.g., to a site in the pancreas or into a brain artery or directly into brain tissue. The compositions may also be administered directly to the target site, e.g., by biolistic delivery to an external or internal target site, like the pancreas or brain. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Pharmaceutically active matter may be present in an effective therapeutic amount, which may be between 0.1 ng and 10 mg/kg body weight per dose; however, doses below or above this exemplary range are envisioned, especially considering the aforementioned factors.

Also provided is one or more compounds of the invention as disclosed herein for use in diagnostic medicine.

Preferred uses in medicine are in nuclear medicine such as nuclear diagnostic imaging, also named nuclear molecular imaging, and/or targeted radiotherapy of diseases associated with an overexpression, preferably of PSMA on the diseased tissue.

Also provided is a compound of the invention as defined herein for use in a method of diagnosing and/or staging cancer, preferably prostate cancer. Prostate cancer is not the only cancer to express PSMA. Nonprostate cancers to demonstrate PSMA expression include breast, lung, colorectal, and renal cell carcinoma. Thus, any compound described herein having a PSMA binding moiety can be used in the diagnosis, imaging or treatment of a cancer having PSMA expression.

Preferred indications are the detection or staging of cancer, such as, but not limited high grade gliomas, lung cancer and especially prostate cancer and metastasized prostate cancer, the detection of metastatic disease in patients with primary prostate cancer of intermediate-risk to high-risk, and the detection of metastatic sites, even at low serum PSA values in patients with biochemically recurrent prostate cancer. Another preferred indication is the imaging and visualization of neoangiogensis.

Also provided is a compound of the invention as defined herein for use in a method of diagnosing and/or staging cancer, preferably prostate cancer.

Also provided is a pharmaceutical or diagnostic composition comprising or consisting of one or more compounds of the invention. The compounds of the invention may be for use as a cancer diagnostic or imaging agent. Accordingly also provided is a method of imaging and/or diagnosing cancer comprising administering a compound of the invention or a composition comprising a compound of the invention. The compounds or compositions of the invention may be for use in the treatment of cancer. The compounds or compositions of the invention may be for use in the diagnosis, imaging or prevention of neoangiogenesis/angiogenesis. The compounds or compositions of the invention may be for use as a cancer diagnostic or imaging agent or for use in the treatment of cancer. The compounds or compositions of the invention may be for use as a cancer diagnostic or imaging agent or for use in the treatment of cancer wherein the cancer is prostate, breast, lung, colorectal or renal cell carcinoma.

The term “treatment”, in relation to the uses of any of the compounds described herein, is used to describe any form of intervention where a compound is administered to a subject suffering from, or at risk of suffering from, or potentially at risk of suffering from the disease or disorder in question. Thus, the term “treatment” covers both preventative (prophylactic) treatment and treatment where measurable or detectable symptoms of the disease or disorder are being displayed.

The term “effective therapeutic amount” (for example in relation to methods of treatment of a disease or condition) refers to an amount of the compound which is effective to produce a desired therapeutic effect.

Any chemical terms are used in their conventional sense (e.g. as defined in the IUPAC Gold Book), unless indicated otherwise.

To the extent that any of the compounds described have chiral centres, the present invention extends to all optical isomers of such compounds, whether in the form of racemates or resolved enantiomers. The invention described herein relates to all crystal forms, solvates and hydrates of any of the disclosed compounds however so prepared. To the extent that any of the compounds disclosed herein have acid or basic centres such as carboxylates or amino groups, then all salt forms of said compounds are included herein. In the case of pharmaceutical uses, the salt should be seen as being a pharmaceutically acceptable salt.

Salts or pharmaceutically acceptable salts that may be mentioned include acid addition salts and base addition salts as well as salt forms arising due to the presence of the chelated nonradioactive or radioactive cation. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Beyond lutetium, further examples of pharmaceutically acceptable salts include acid addition salts derived from mineral acids and organic acids, and salts derived from metals such as sodium, magnesium, potassium and calcium.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), a-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g.(+)-L-tartaric), thiocyanic, undecylenic and valeric acids.

Also encompassed are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents may include water, alcohols (such as ethanol, isopropanol and butanol) and dimethylsulfoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates. For a more detailed discussion of solvates and the methods used to make and characterise them, see Bryn et al, Solid-State Chemistry of Drugs, Second Edition, published by SSCI, Inc of West Lafayette, IN, USA, 1999, ISBN 0-967-06710-3.

The compounds of the invention may contain one or more isotopic substitutions, and a reference to a particular element includes within its scope all isotopes of the element. For example, a reference to hydrogen includes within its scope 1H, 2H (D), and 3H (T). Similarly, references to carbon and oxygen include within their scope respectively 12C, 13C and 14C and 160 and 180. In an analogous manner, a reference to a particular functional group also includes within its scope isotopic variations, unless the context indicates otherwise. For example, a reference to an alkyl group such as an ethyl group or an alkoxy group such as a methoxy group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group) or a methoxy group in which all three hydrogen atoms are in the deuterium isotopic form (a trideuteromethoxy group). The isotopes may be radioactive or non-radioactive.

### Methods

### General Information

Analytical and preparative high-performance liquid chromatography (H PLC) was performed using Shimadzu gradient systems (Neufahrn, Germany) equipped with a SPD-20A UV/Vis detector. The columns for analytical (MultoKrom 100C18, 150×4.6 mm, 5 μm), radio-analytical (Multospher 100RP18, 125×4.6 mm, 5 μm) and preparative (MultoKrom 100C18, 250×20 mm, 5 μm) HPLC were purchased from CS Chromatographie Service (Langerwehe, Germany). Eluents for all HPLC operations were water (solvent A) and acetonitrile with 2 vol.-% water (solvent B), both containing 0.1 vol. % trifluoroacetic acid (TFA). Radioactivity was detected via a HERM LB 500 Nal detector (Berthold Technologies, Bad Wildbad, Germany). Radio-thin layer chromatography (TLC) was carried out with a Scan-RAM detector (LabLogic Systems, Sheffield, United Kingdom). Electrospray ionization-mass spectra were acquired on an expression^{L} CMS (Advion, Harlow, UK).

### Synthesis of PSMA Ligands

The uncomplexed radiohybrid ligands rhPSMA-7.1, -7.2, -7.3 and -7.4 were prepared applying a mixed solid phase/solution phase synthetic strategy, according to the literature protocols (Wurzer A, et al. EJNMMI Res. 2020; 10: 149). rhPSMA-10.1 and -10.2 were obtained in analogy to the rhPSMA-7 isomers, by substitution of the DOTA-GA chelator by DOTA. PSMA I&T was prepared according to the published procedure (Weineisen M, et al. J Nucl Med. 2015; 56: 1169-1176) and PSMA-617 was purchased from MedChemExpress LLC (Monmouth Junction, USA). For complexation with non-radioactive lutetium for in vitro studies, a 2 mM solution of the PSMA inhibitor (1.0 eq.) in DMSO was combined with a 20 mM aqueous solution of LuCl₃ (2.5 eq.) and heated to 95° C. for 30 min. Analytical data of the Lu-chelated PSMA ligands is provided below.

### Radiolabeling

Radiolabeling with Lu-177 was performed according to the established procedures for PSMA-targeted ligands (Benesova M, et al. J Nucl Med. 2015; 56: 914-920; Weineisen M, et al. J Nucl Med. 2015; 56: 1169-1176). Briefly, the precursor (1.0 nmol, 10 μL, 0.1 mM in DMSO) was added to 10 μL of 1.0 M aqueous NaOAc buffer (pH 5.5). Subsequently, 20 to 50 MBq ¹⁷⁷LuCl₃ (Molar Activity>3000 GBq/mg, 740 MBq/mL, 0.04 M HCl, ITM, Garching, Germany) were added and the mixture was filled up to 100 μL with 0.04 M HCl. The reaction mixture was heated for 20-30 min at 90° C. and the radiochemical purity (RCP) was determined using radio-HPLC and radio-TLC with 0.1 M sodium citrate buffer on iTLC-SG chromatography paper (Agilent, Santa Clara, USA) and 1.0 M NH₄ OAc/DMF buffer (1/1; v/v) on TLC Silica gel 60 F₂₅₄ plates (Merck Millipore, Burlington, USA).

### Lipophilicity

Approximately 1 MBq of the ¹⁷⁷Lu-labeled PSMA ligand was dissolved in 1 mL of a 1:1 mixture (v/v) of phosphate-buffered saline (PBS, pH 7.4) and n-octanol (n=6). After vigorous mixing of the suspension for 3 min, the vial was centrifuged at 15000×g for 3 min and 100 μL aliquots of both layers were measured in a γ-counter. Finally, the ratio of the radioactivity detected in the n-octanol sample and the PBS buffer was calculated and expressed as distribution ratio log D_{7.4.}

### Binding to Human Serum Albumin (HSA)

Binding of ¹⁷⁷Lu-labeled ligands to HSA was assessed by albumin mediated size exclusion chromatography (AMSEC). A gel filtration size exclusion column (Superdex 75 Increase 10/300 GL, GE Healthcare, Uppsala, Sweden) was used with HSA buffer at physiological concentration (700 μM, Biowest, Nuaillé, France) as mobile phase (constant flow rate of 0.8 mL/min at room temperature). For calibration purposes, a commercially available set of proteins (GE Healthcare, Buckinghamshire, UK) was used. Under these chromatographic conditions, the retention time of a radioligand (1.0 MBq, 10-20 GBq/μmol) depends on the extent of HSA/ligand interactions and thus, by means of the calibration with the aforementioned set of proteins, can be translated into an AMW (expressed in kDa) as a parameter allowing to quantify the extent of HSA binding. The detection window ranges between 2.3 kDa ([¹⁸F]Fluoride; no HSA interaction) and 70.2 kDa (experimental molecular weight of HSA; maximum HSA interaction).

### Affinity Determinations (ICH) and Internalization Studies

Competitive binding studies were determined on LNCaP cells (1.5×10⁵ cells in 1 mL/well) after incubation at 4° C. for 1 h, using (((S)-1-carboxy-5-(4-([¹²⁵I]iodo)benzamido)pentyl)carbamoyl)-L-glutamic acid ([¹²⁵I]BA)KuE; 0.2 nM/well) as reference radioligand (n=3). Internalization studies of the radiolabeled ligands (1.0 nM/well) were performed on LNCaP cells (1.25×10⁵ cells in 1 mL/well) at 37° C. for 1 h and accompanied by ([¹²⁵I]BA)KuE (0.2 nM/well), as reference. Data were corrected for non-specific binding and normalized to the specific internalization observed for the reference (n=3). A detailed description of the experimental procedures was previously published (Wurzer A, et al. J Nucl Med. 2020; 61: 735-742).

### In Vivo Experiments

All animal experiments were conducted in accordance with general animal welfare regulations in Germany (German animal protection act, as amended on 18 May 2018, Art. 141 G v. 29 Mar. 2017 I 626, approval no. 55.2-1-54-2532-71-13) and the institutional guidelines for the care and use of animals. LNCaP tumor xenografts were established in 6-8 weeks old male CB-17 SCID mice as described previously (Wurzer A, et al. J Nucl Med. 2020; 61: 735-742). Biodistribution Studies. The ¹⁷⁷Ludabeled PSMA inhibitors (2-5 MBq; 0.1 nmol) were injected under isoflurane anesthesia into the tail vein of LNCaP tumor-bearing male CB-17 SCID mice that were sacrificed 24 h post injection (p.i.) (n=4-5). Selected organs were removed, weighed, and measured in a counter. All rhPSMA ligands were evaluated in mice during the same time period (Q1/2020), whereas ¹⁷⁷Ludabeled PSMA-617 and PSMA I&T (Wirtz M, et al. EJNMMI Res. 2018; 8: 84) were assessed previously using the identical cell line, mouse model and experimental procedure.

μSPECT/CT Imaging. Static images of ¹⁷⁷Lu-labeled inhibitors were recorded of sacrificed mice, 24 h p.i. directly after blood collection, with an acquisition time of 45 min using the HE-GP-RM collimator and a step-wise multi-planar bed movement. For imaging studies, a MlLabs VECTor4 small-animal SPECT/PET/OI/CT from MlLabs (Utrecht, Netherlands) was applied. Data were reconstructed using the MILabs-Rec software (version 10.02) and PMOD4.0 software (PMOD TECHNOLOGIES LLC, Zurich, Switzerland).

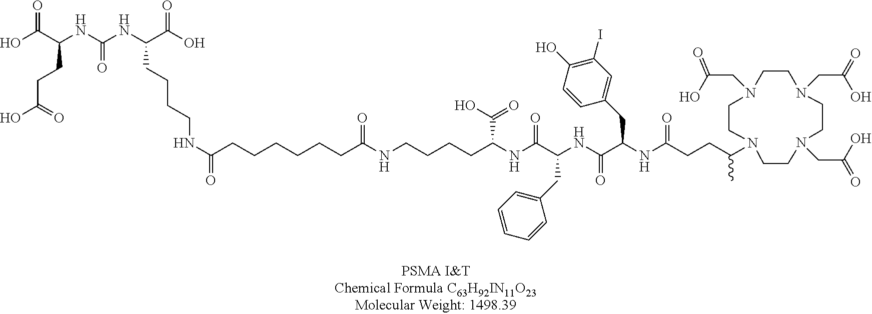 [SMILES: CC(CCC(=O)N[C@H](Cc1ccc(O)c(I)c1)C(=O)N[C@H](Cc1ccccc1)C(=O)N[C@H](CCCCNC(=O)CCCCCCC(=O)NCCCC[C@H](NC(=O)N[C@@H](CCC(=O)O)C(=O)O)C(=O)O)C(=O)O)N1CCN(CC(=O)O)CCN(CC(=O)O)CCN(CC(=O)O)CC1]

### Results

### Synthesis and Radiolabeling

Uncomplexed PSMA ligands were obtained via a solid phase/solution phase synthetic strategy with chemical purities >97% as determined by HPLC (absorbance at 220 nm). Identity was confirmed by mass spectrometry. Complexation with 2.5-fold molar excess LuCl₃ lead to quantitative formation of the respective Lu-PSMA ligands, which were used for in vitro studies. ¹⁷⁷Lu-labeling of PSMA ligands according to standard manual procedures resulted in a RCP >95%, determined by radio-HPLC and radio-TLC.

PSMA-10 (1) in its free chelator form was synthesized according to WO2020157184A1 and WO2020157177A1. Briefly, the tent-butyl protected chelator, DOTA(tBu)₃ was conjugated to the free N-terminus with a mixture of HOAt (2.0 eq.), TBTU (2.0 eq.) and 2,4,6-trimethylpyridine (6.7 eq.) for 2 h in DMF. Cleavage from the resin and deprotection of acid labile protecting groups was performed in TFA for 6 h. After RP-HPLC-based purification, rhPSMA-10 (1) (18%) was obtained as a colorless solid. RP-HPLC (10 to 70% B in 15 min): t_{R}=9.9 min, K′=3.95. Calculated monoisotopic mass (C₆₀H₉₅FN₁₂O₂₃Si): 1398.6; found: m/z=1399.6 [M+H]⁺, 700.6 [M+2H]²⁺.

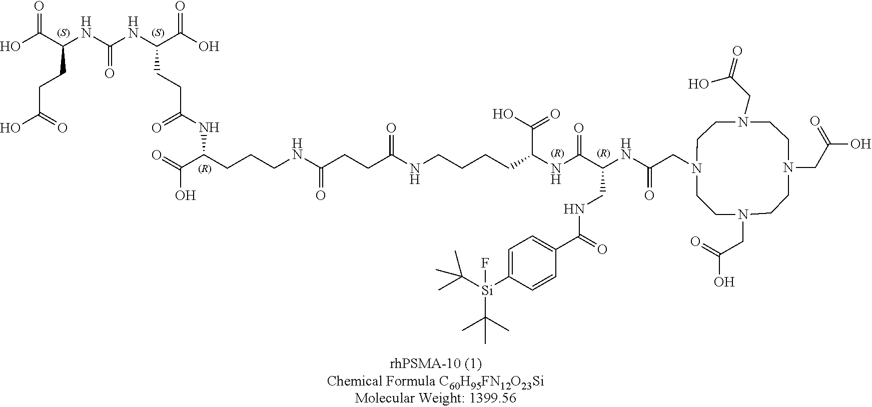

Preparation of ^{nat}Lu-1 and [¹⁷⁷Lu]Lu-1 followed similar procedures to those conducted in the literature (WO2019/020831). The corresponding ^{nat}Lu-complexes were prepared from a 2 mM solution of the PSMA inhibitor (1.0 eq.) in DMSO with a 20 mM aqueous solution of LuCl₃ (2.5 eq.), heated to 95 ° C. for 30 min. After cooling, the ^{nat}Lu-chelate formation was confirmed using RP-HPLC and MS.

¹⁹F-^{nat}Lu-rhPSMA-10 (1): RP-HPLC (10 to 70% B in 15 min): t_{R}=9.9 min, K′=3.95. Calculated monoisotopic mass (C₆₀H₉₂FLu₁₂O₂₃Si): 1570.6; found: m/z=1572.2 [M+H]⁺, 786.6 [M+2H]²⁺.

### In Vitro Characterization

Results of the in vitro evaluation of all rhPSMAs and the well-established reference inhibitors PSMA-617 (Benesova M, et al. J Nucl Med. 2015; 56: 914-920) and PSMA I&T (Weineisen M, et al. J Nucl Med. 2015; 56: 1169-1176) are summarized in FIG. 2 and Table 1. PSMA binding affinity (IC₅₀; FIG. 2, A) was high and in the low nanomolar range for all [^{nat}Lu, ¹⁹F]rhPSMA ligands (range: 2.8±0.5 to 3.6±0.6 nM) and the two state-of-the art reference inhibitors ([^{nat}Lu]PSMA I&T: 4.2±0.8 nM, [^{nat}Lu]PSMA-617: 3.3±0.2 nM).

Slight differences between the ligands were found for the PSMA-mediated internalization into LNCaP cells (1 h, 37° C.), which is expressed as a percentage of the specific internalization of the reference ligand ([125I]BA)KuE (FIG. 2B). While [¹⁷⁷Lu, ¹⁹F]rhPSMA-7.1 and [¹⁷⁷Lu]PSMA I&T showed the lowest internalization rates with values of 137±6% and 145±14%, respectively, the other isomers showed an approximately 1.4-fold higher internalization (range: 177±15 to 206±8%), similar to that of [¹⁷⁷Lu]PSMA-617 (203±10%).

The ¹⁷⁷Ludabeled rhPSMA-7 isomers as well as the reference PSMA I&T and PSMA-617 demonstrated a high and similar hydrophilicity, expresses as partition-coefficient (log D_{7.4}; n-octanol and PBS pH 7.4) with values between -4.1±0.1 and -4.3±0.3. The DOTA-conjugates [¹⁷⁷Lu, ¹⁹F]rhPSMA-10.1 and -10.2, showed a slightly lower hydrophilicity with a log D_{7.4} value of −3.8 (FIG. 2C).

The apparent molecular weight of the tracers was determined to compare the relative HSA-binding strength of the inhibitors. Interestingly, remarkable differences were found for the AMWs of the state-of-the-art references and even among the single isomers of ¹⁷⁷Lu-labeled rhPSMA-7 and rhPSMA-10, respectively (FIG. 2D). While [¹⁷⁷Lu]PSMA I&T showed lowest HSA interaction (AMW=5.3 kDa) followed by [¹⁷⁷Lu]PSMA-617 (AMW=13.7 kDa), all radiohybrid inhibitors demonstrated an at least 1.5-fold higher AMW with values between 21.8 and 35.7 kDa. Among the radiohybrids, the two DOTA-conjugates [¹⁷⁷Lu, ¹⁹F]rhPSMA-10.1 and -10.2 showed the lowest AMW (25.1 kDa and 21.8 kDa), respectively, while D-Dap-configured [¹⁷⁷Lu, ¹⁹F]rhPSMA-7.1 (MW=26.3 kDa) and [¹⁷⁷Lu, ¹⁹F]rhPSMA-7.3 (MW=30.4kDa) showed the lowest AMWs within the rhPSMA-7 series (AMWs of L-Dap-comprising isomers: [¹⁷⁷Lu, ¹⁹F]rhPSMA-7.2=31.7 kDa and [¹⁷⁷Lu, ¹⁹F]rhPSMA-7.4=35.7 kDa).

### In Vivo Characterization

### Biodistribution Studies

Overall, the comparative biodistribution study of the ¹⁷⁷Lu-labeled PSMA-ligands in LNCaP tumor-bearing mice at 24 h p.i. revealed a quite similar distribution pattern with high tumor uptake, fast excretion from background organs, but varying degree of activity retention in the kidneys (FIG. 3, Table 2 and 3).

Highest activity retention in the kidneys was found for [¹⁷⁷Lu]PSMA I&T (15.9±12.0% ID/g), whereas [¹⁷⁷Lu, ¹⁹F]rhPSMA-10.1 (2.4±01.6% ID/g) demonstrated fastest renal clearance. Kidney uptake of [¹⁷⁷Lu, ¹⁹F]rhPSMA-7.3 was found to be 11.4±1.4% ID/g, thus showing slower renal clearance than [¹⁷⁷Lu, ¹⁹F]rhPSMA-10.1. These differences are also well illustrated in the ASPECT/CT images (see FIG. 5). Tumor uptake was highest for all [¹⁷⁷Lu, ¹⁹F]rhPSMA-7 isomers and in the range of 11.6-12.7% ID/g, followed by [¹⁷⁷Lu, ¹⁹F]rhPSMA-10.2 (10.5±3.3% ID/g) and -10.1 (9.8±0.3% ID/g), whereas the state-of-the-art references ¹⁷⁷Lu-labeled PSMA I&T (4.1±1.1* ID/g) exhibited a lower tumor uptake.

### Tumor-to-Organ Ratios

Interestingly, all radiohybrid inhibitors are cleared from the blood pool and background tissues with a kinetics that more resembles that of small molecules than that of larger proteins - despite their extensive binding to HSA. Amongst all radiohybrids, the highest tumor/blood and tumor/kidney ratio was found for [¹⁷⁷Lu, ¹⁹F]rhPSMA-10.1 (T/blood: 9117, T/kidney: 5.5), followed by [¹⁷⁷Lu, ¹⁹F]rhPSMA-7.3 (T/blood: 4255, T/kidney: 1.64). While [¹⁷⁷Lu]PSMA I&T (T/Blood: 1288; T/Kidney: 0.6) exhibited rather slow excretion in mice.

All inhibitors showed potent binding to PSMA-expressing LNCaP cells with affinities in the low nanomolar range and high internalization rates. Surprisingly, most pronounced differences were identified regarding the HSA-related AMW. While [¹⁷Lu, ¹⁹F]rhPSMA-7 isomers demonstrated the highest AMW and thus strongest HSA-interactions, [¹⁷⁷Lu, ¹⁹F]rhPSMA-10.1 showed an AMW lower than [¹⁷⁷Lu, ¹⁹F]rhPSMA-7.3 but higher than the ¹⁷⁷Lu-labeled references PSMA I&T and PSMA-617. In biodistribution studies [¹⁷⁷Lu, ¹⁹F]rhPSMA-10.1 exhibited the lowest kidney uptake and fastest excretion from the blood pool of all rhPSMA ligands, while preserving a high tumor accumulation.

### Analytical Data of Lu-Complexed PSMA Inhibitors

[^{nat}Lu, ¹⁹F]rhPSMA-7.1: RP-HPLC (10 to 70% B in 15 min): t_{R}=9.7 min, K′=3.85. Calculated monoisotopic mass (C₆₃H₉₆FLuN₁₂O₂₅Si): 1642.6; found: m/z=1643.5 [M+H]⁺, 822.5 [M+2H]²⁺.

[^{nat}Lu, ¹⁹F]rhPSMA-7.2: RP-HPLC (10 to 70% B in 15 min): t_{R}=9.4 min, K′=3.70. Calculated monoisotopic mass (C₆₃H₉₆FLuN₁₂O₂₅Si): 1642.6; found: m/z=1642.9 [M+H]⁺, 822.0 [M+2H]²⁺.

[^{nat}Lu, ¹⁹F]rhPSMA-7.3: RP-HPLC (10 to 70% B in 15 min): t_{R}=9.6 min, K′=3.80. Calculated monoisotopic mass (C₆₃H₉₆FLuN₁₂O₂₅Si): 1642.6; found: m/z=1643.4 [M+H]⁺, 822.3 [M+2H]²⁺.

[^{nat}Lu, ¹⁹F]rhPSMA-7.4: RP-HPLC (10 to 70% B in 15 min): t_{R}=9.6 min, K′=3.80. Calculated monoisotopic mass (C₆₃H₉₆FLuN₁₂O₂₅Si): 1642.6; found: m/z=1643.0 [M+H]⁺, 822.3 [M+2H]²⁺.

[^{nat}Lu, ¹⁹F]rhPSMA-10.1: RP-HPLC (10 to 70% B in 15 min): t_{R}=9.9 min, K′=3.95. Calculated monoisotopic mass (C₆₀H₉₂FLuN₁₂O₂₃Si): 1570.6; found: m/z=1571.8 [M+H]⁺, 786.2 [M+2H]²⁺.

[^{nat}Lu, ¹⁹F]rhPSMA-10.2: RP-HPLC (10 to 70% B in 15 min): t_{R}=9.6 min, K′=3.80. Calculated monoisotopic mass (C₆₀H₉₂FLuN₁₂O₂₃Si): 1570.6; found: m/z=1571.9 [M+H]⁺, 786.6 [M+2H]²⁺.

[^{nat}Lu]PSMA-I&T: RP-HPLC (10 to 70% B in 15 min): t_{R}=7.2 min, K′=3.32. Calculated monoisotopic mass (C₆₃H₈₉ILuN₁₁O₂₃): 1669.5; found: m/z=1670.5 [M+H]⁺, 1113.8 [2M+3H]³⁺.

[^{nat}Lu]PSMA-617: RP-HPLC (10 to 70% B in 15 min): t_{R}=6.5 min, K′=2.82. Calculated monoisotopic mass (C₄₉H₆₈LuN₉O₁₆): 1213.4; found: m/z=1213.6 [M+H]⁺, 607.5 [M+2H]²⁺.

TABLE 1
Binding affinities (IC₅₀ [nM], 1 h, 4° C.) of [^{nat}Lu, ¹⁹F]rhPSMA-7.1 to -7.4 (n =
3), [^{nat}Lu, ¹⁹F]rhPSMA-10.1, -10.2 (n = 3) and the references [^{nat}Lu]PSMA-617
and [^{nat}Lu]PSMA-I&T (n = 3); B) PSMA-mediated internalization of [¹⁷⁷Lu,
¹⁹F]rhPSMA-7.1 to -7.4 (n = 3), [¹⁷⁷Lu, ¹⁹F]rhPSMA-10.1, -10.2 (n = 3) and the
references [¹⁷⁷Lu]PSMA-617 and [¹⁷⁷Lu]PSMA I&T (n = 3) by LNCaP cells (1 h,
37° C.) as a percentage of the reference ligand ([¹²⁵I]BA)KuE); Lipophilicity of
[¹⁷⁷Lu, ¹⁹F]rhPSMA-7.1 to -7.4 (n = 6), [¹⁷⁷Lu, ¹⁹F]rhPSMA-10.1, -10.2 (n = 6)
and the references [¹⁷⁷Lu]PSMA-617 and [¹⁷⁷Lu]PSMA I&T (n = 6), expressed as
partition coefficient (log D_{7.4}) using the n-octanol/PBS (pH 7.4) distribution system;
Apparent molecular weight (AMW) of [¹⁷⁷Lu, ¹⁹F]rhPSMA-7.1 to -7.4, [¹⁷⁷Lu,
¹⁹F]rhPSMA-10.1, -10.2 and the references [¹⁷⁷Lu]PSMA-617 and [¹⁷⁷Lu]PSMA
I&T, as determined by human serum albumin-related determined on a size exclusion
chromatography with HSA in the mobile phase.

| | [¹⁷⁷Lu, ¹⁹F] | [¹⁷⁷Lu, ¹⁹F] | [¹⁷⁷Lu, ¹⁹F] | [¹⁷⁷Lu, ¹⁹F] |
|---|---|---|---|---|
| Compound | rhPSMA-7.1 | rhPSMA-7.2 | rhPSMA-7.3 | rhPSMA-7.4 |
| IC₅₀ [nM] | 3.11 ± 0.64 | 2.88 ± 1.06 | 3.29 ± 1.00 | 3.06 ± 1.51 |
| Internalization | 137.4 ± 5.8 | 197.3 ± 15.0 | 184.4 ± 11.8 | 190.4 ± 10.5 |
| [% IBA-KuE] | | | | |
| logD_{7.4} | −4.27 ± 0.24 | −4.25 ± 0.29 | −4.12 ± 0.11 | −4.10 ± 0.14 |
| MWₐₚₚ [kDa] | 26.3 | 31.7 | 30.4 | 35.7 |

| | [¹⁷⁷Lu, ¹⁹F] | [¹⁷⁷Lu, ¹⁹F] | [¹⁷⁷Lu] | [¹⁷⁷Lu] |
|---|---|---|---|---|
| Compound | rhPSMA-10.1 | rhPSMA-10.2 | PSMA-617 | PSMA I&T |
| IC₅₀ [nM] | 2.76 ± 0.51 | 3.61 ± 0.59 | 3.27 ± 0.19 | 4.17 ± 0.78 |
| Internalization | 177.4 ± 14.6 | 205.6 ± 8.3 | 203.2 ± 10.1 | 145.4 ± 13.8 |
| [% IBA-KuE] | | | | |
| logD_{7.4} | −3.78 ± 0.06 | −3.83 ± 0.10 | −4.1 ± 0.1 | −4.1 ± 0.1 |
| MWₐₚₚ [kDa] | 25.1 | 21.8 | 13.7 | 5.3 |

TABLE 2
Complete dataset of ex vivo biodistribution studies of the compounds
[¹⁷⁷Lu]rhPSMA-7.3 (n = 5), [¹⁷⁷Lu]rhPSMA-10.1 (n = 5) and
[¹⁷⁷Lu]PSMA-I&T (n = 5) at 24 h p.i. in male tumor-bearing
CB17-SCID mice. Data expressed as percentage of the injected
dose per gram (% ID/g), mean ± standard deviation.

| Activity | | | | | | |
|---|---|---|---|---|---|---|
| accumulation | | | | | | |
| [% ID/g] | [¹⁷⁷Lu]rhPSMA-7.3 | | [¹⁷⁷Lu]rhPSMA-10.1** | | [¹⁷⁷Lu]PSMA-I&T | |
| (complete | 24 h p.i., n = 5 | | 24 h p.i., n = 5 | | 24 h p.i., n = 5 | |
| dataset) | mean | SD | mean | SD | mean | SD |
| blood | 0.0046 | 0.0020 | 0.0019 | 0.0012 | 0.0061 | 0.0027 |
| heart | 0.0333 | 0.0030 | 0.0192 | 0.0061 | 0.0208 | 0.0068 |
| lung | 0.0768 | 0.0179 | 0.0346 | 0.0163 | 0.1138 | 0.0228 |
| liver | 0.2352 | 0.0382 | 0.1631 | 0.0561 | 0.0553 | 0.0263 |
| spleen | 0.5817 | 0.1783 | 0.1828 | 0.0408 | 1.2047 | 1.0492 |
| pancreas | 0.0241 | 0.0042 | 0.0155 | 0.0067 | 0.0295 | 0.0256 |
| stomach | 0.0434 | 0.0177 | 0.0388 | 0.0243 | 0.0326 | 0.0125 |
| intestine | 0.1001 | 0.0653 | 0.0642 | 0.0534 | 0.0507 | 0.0290 |
| kidney | 11.3738 | 1.4171 | 2.4219 | 1.5547 | 15.8527 | 11.9512 |
| adrenals | 0.4233 | 0.0855 | 0.0983 | 0.0607 | 1.3590 | 0.4626 |
| muscle | 0.0094 | 0.0032 | 0.0053 | 0.0024 | 0.0051 | 0.0033 |
| bone | 0.0307 | 0.0067 | 0.0298 | 0.0065 | 0.0209 | 0.0059 |
| tumor | 18.1740 | 4.4592 | 11.5563 | 3.7945 | 7.5780 | 2.6785 |
| parotid gl. | 0.1068 | 0.0284 | 0.0551 | 0.0106 | 0.1346 | 0.0814 |
| submand. gl. | 0.0644 | 0.0134 | 0.0338 | 0.0082 | 0.0581 | 0.0277 |

TABLE 3
Tumor-to-organ ratios obtained from the complete dataset of ex
vivo biodistribution studies of the compounds [¹⁷⁷Lu]rhPSMA-7.3
(n = 5), [¹⁷⁷Lu]rhPSMA-10.1 (n = 5) and [¹⁷⁷Lu]PSMA-I&T
(n = 5) at 24 h p.i. in male tumor-bearing CB17-SCID mice.
Ratios are calculated individually for each mouse and expressed
as mean ± standard deviation.

| Tumor-to-organ | [¹⁷⁷Lu]rhPSMA-7.3 | | [¹⁷⁷Lu]rhPSMA-10.1** | | [¹⁷⁷Lu] PSMA-I&T | |
|---|---|---|---|---|---|---|
| ratio | 24 h p.i., n = 5 | | 24 h p.i., n = 5 | | 24 h p.i., n = 5 | |
| (complete dataset) | mean | SD | mean | SD | mean | SD |
| blood | 4255.56 | 1151.85 | 9117.20 | 6829.96 | 1288.54 | 136.30 |
| heart | 542.49 | 100.85 | 641.37 | 299.14 | 366.06 | 61.92 |
| lung | 239.97 | 46.92 | 404.28 | 263.36 | 65.08 | 10.97 |
| liver | 78.11 | 18.61 | 72.52 | 21.93 | 155.09 | 63.81 |
| spleen | 34.32 | 14.95 | 63.66 | 21.20 | 9.06 | 4.56 |
| pancreas | 754.34 | 110.20 | 797.72 | 301.51 | 336.33 | 150.72 |
| stomach | 453.70 | 147.52 | 351.52 | 133.65 | 236.69 | 41.44 |
| intestine | 254.02 | 173.89 | 250.87 | 133.39 | 191.31 | 116.16 |
| kidney | 1.64 | 0.59 | 5.52 | 1.91 | 0.61 | 0.23 |
| adrenals | 44.34 | 13.03 | 137.57 | 52.77 | 5.62 | 1.33 |
| muscle | 2123.58 | 888.96 | 2431.90 | 1097.64 | 1964.92 | 1251.14 |
| bone | 625.98 | 243.08 | 389.88 | 128.82 | 358.26 | 47.36 |
| parotid gl. | 176.66 | 42.76 | 206.18 | 43.15 | 63.21 | 14.84 |
| submand. gl. | 290.08 | 79.53 | 344.45 | 97.54 | 136.76 | 19.38 |

### BRIEF DESCRIPTION OF THE FIGURES

FIG. 1: (A) The rhPSMA-7 isomers are differing in the stereoconfiguration of the diaminopropionic acid branching unit (D-Dap or L-Dap) and the glutamic acid pendant arm at the DOTA-GA-chelator (R- or S-DOTA-GA). (B) rhPSMA-10.1 (D-Dap) and rhPSMA-10.2 (L-Dap), both equipped with the DOTA chelator, are also differing in the stereoconfiguration of the branching unit (D-Dap or L-Dap). The well-established PSMA-addressing ligands PSMA-617 (C) and PSMA I&T (D) served as reference compounds (Benesova M, et al. J Nucl Med. 2015; 56: 914-920; Weineisen M, et al. J Nucl Med. 2015; 56: 1169-1176).

FIG. 2: A) Binding affinities (IC₅₀ [nM], 1 h, 4° C.) of [^{nat}Lu, ¹⁹F]rhPSMA-7.1 to -7.4 (white; n=3), [^{nat}Lu, ¹⁹F]rhPSMA-10.1, -10.2 (black/white stripes; n=3) and the references [^{nat}Lu]PSMA-617 and [^{nat}Lu]PSMA-I&T (black; n=3); B) PSMA-mediated internalization of [¹⁷⁷Lu, ¹⁹F]rhPSMA-7.1 to -7.4 (white; n=3), [¹⁷⁷Lu, ¹⁹F]rhPSMA-10.1, -10.2 (black/white stripes; n=3) and the references [¹⁷⁷Lu]PSMA-617 and [¹⁷⁷Lu]PSMA I&T (black; n=3) by LNCaP cells (1 h, 37° C.) as a percentage of the reference ligand ([¹²⁵I]BA)KuE); C) lipophilicity of [¹⁷⁷Lu, ¹⁹F]rhPSMA-7.1 to -7.4 (white; n=6), [¹⁷⁷Lu, ¹⁹F]rhPSMA-10.1, -10.2 (black/white stripes; n=6) and the references [¹⁷⁷Lu]PSMA-617 and [¹⁷⁷Lu]PSMA I&T (black, n=6), expressed as partition coefficient (log D_{7.4} in n-octanol/PBS pH 7.4); D) apparent molecular weight (AMW) of [¹⁷⁷Lu, ¹⁹F]rhPSMA-7.1 to -7.4 (white), [¹⁷⁷Lu, ¹⁹F]rhPSMA-10.1, -10.2 (black/white stripes) and the references [¹⁷⁷Lu]PSMA-617 and [¹⁷⁷Lu]PSMA I&T (black), as determined by human serum albumin-related determined on a size exclusion chromatography with HSA in the mobile phase.

FIG. 3: Biodistribution of [¹⁷⁷Lu, ¹⁹F]rhPSMA-7.3, [¹⁷⁷Lu, ¹⁹F]rhPSMA-10.1 and the reference [¹⁷⁷Lu]PSMA I&T at 24 h p.i. in male LNCaP tumor-bearing SCID mice. Data are expressed as a percentage of the injected dose per gram [% ID/g], mean±standard deviation (n=5) (data plotted from table 2).

FIG. 4: Tumour to organ ratio of [¹⁷⁷Lu, ¹⁹F]rhPSMA-7.3, [¹⁷⁷Lu, ¹⁹F]rhPSMA-10.1 and the reference [¹⁷⁷Lu]PSMA I&T at 24 h p.i. in male LNCaP tumor-bearing SCID mice. Data are expressed as a percentage of the injected dose per gram [% ID/g], mean±standard deviation (n=5) (data plotted from table 3). FIG. 4a shows a large scale axis and 4b a small scale axis. FIG. 4b shows that the tumour to kidney ratio for [¹⁷⁷Lu, ¹⁹F]rhPSMA-10.1 (5.52 SD 1.91) is greater than either 7.3 or I&T.

FIG. 5: Static ASPECT/CT images (maximum intensity projections) of ¹⁷⁷Lu-labeled rhPSMA-7.3, rhPSMA-7.1 and rhPSMA-10.1 in LNCaP tumor-bearing mice, sacrificed 24 h p.i. and imaged directly after blood collection, with an acquisition time of 45 min on a VECTor4 small-animal SPECT/PET/01/CT. Tumor weight and tracer uptake in the tumor (in percent of the injected dose/gram, [%ID/g]) were determined from subsequent biodistribution studies.

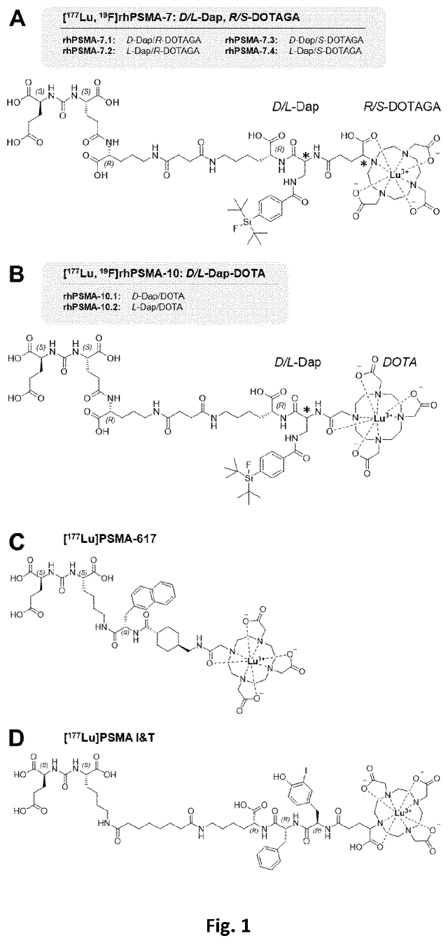

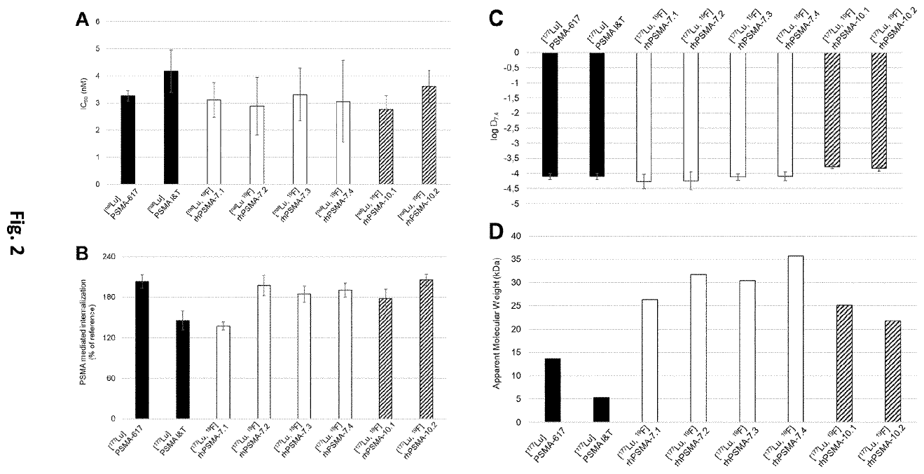

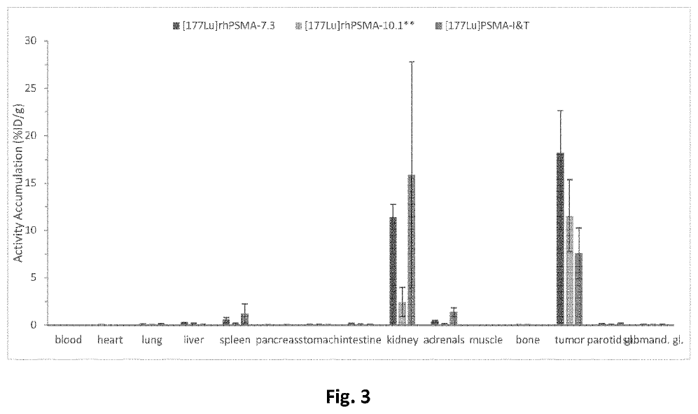

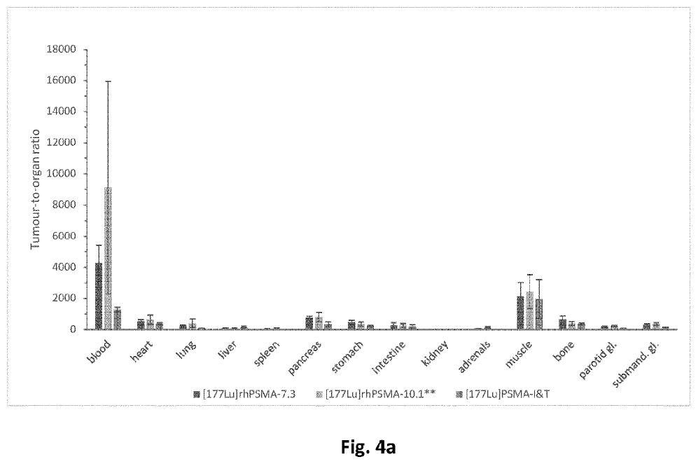

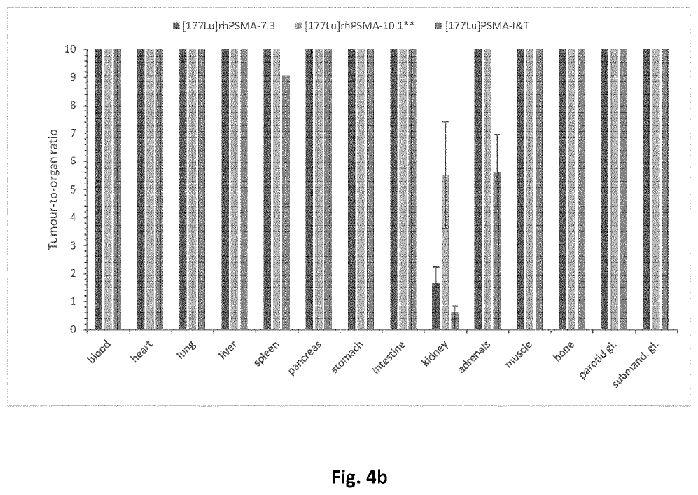

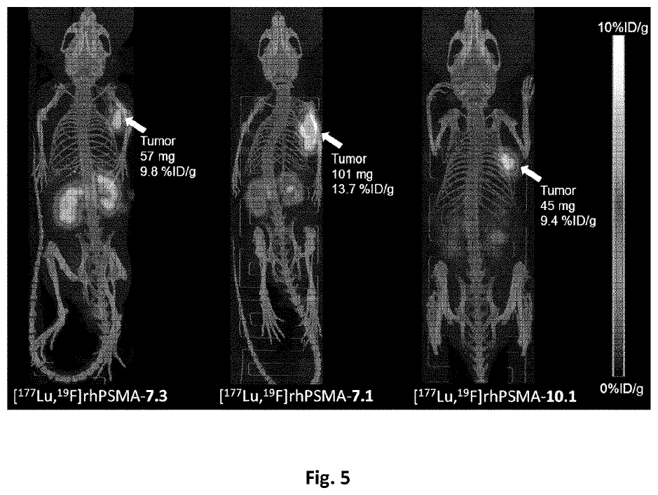

### SUMMARY

The four isomers of [¹⁷⁷Lu, ¹⁹F]rhPSMA-7 ([¹⁷⁷Lu, ¹⁹F]rhPSMA-7.1, -7.2, -7.3 and -7.4) were compared to the state-of-the-art compounds [¹⁷⁷Lu]PSMA I&T and [¹⁷⁷Lu]PSMA-617 and the novel radiohybrid inhibitors [¹⁷⁷Lu, ¹⁹F]rhPSMA-10.1 and -10.2. The comparative evaluation comprised affinity studies (IC₅₀) and internalization experiments on LNCaP cells, as well as lipophilicity measurements. Biodistribution studies and ASPECT imaging was performed in LNCaP-tumor bearing CB-17 SCID mice at 24 h post injection.

In comparative biodistribution studies pronounced different kidney uptake values were observed. Whereas our internal reference D-Dap-S-DOTAGA-configured [¹⁷⁷Lu, ¹⁹F]rhPSMA-7.3 showed a kidney uptake of 11.4±1.4% ID/g at 24 h p.i., the uptake of the D-Dap-DOTA derivative [¹⁷⁷Lu, ¹⁹F]rhPSMA-10.1 reached only 20% of that value (2.4±01.6% ID/g).

Regarding important non-target organs like liver, muscle and heart, all inhibitors demonstrated almost identical and complete clearance 24 h p.i.. Even though only low activity levels were found in the blood pool for all inhibitors, [¹⁷⁷Lu, ¹⁹F]rhPSMA-10.1 showed the best clearance of all investigated PSMA ligands, which is also expressed by the highest tumor-to-blood ratio (T/blood: 9117): 2-times higher value compared to [¹⁷⁷Lu, ¹⁹F]rhPSMA-7.3 and 7-times higher when compared to [¹⁷⁷Lu]PSMA-617.

Results: ¹⁷⁷Lu-labeling of radiohybrids was carried out according to the established procedures for the currently established PSMA-targeted ligands. All inhibitors showed potent binding to PSMA-expressing LNCaP cells with affinities in the low nanomolar range and high internalization rates. Surprisingly, most pronounced differences were identified regarding the HSA-related AMW. While [¹⁷⁷Lu, ¹⁹F]rhPSMA-7 isomers demonstrated the highest AMW and thus strongest HSA-interactions, [¹⁷⁷Lu, ¹⁹F]rhPSMA-10.1 showed an AMW lower than [¹⁷⁷Lu, ¹⁹F]rhPSMA-7.3 but higher than the ¹⁷⁷Ludabeled references PSMA I&T and PSMA-617. In biodistribution studies [¹⁷⁷Lu, ¹⁹F]rhPSMA-10.1 exhibited the lowest kidney uptake and fastest excretion from the blood pool of all rhPSMA ligands, while preserving a high tumor accumulation. Thus compound rhPSMA-10.1 has emerged as a preferred candidate when compared to other related compounds.

1. A compound of Formula (1): 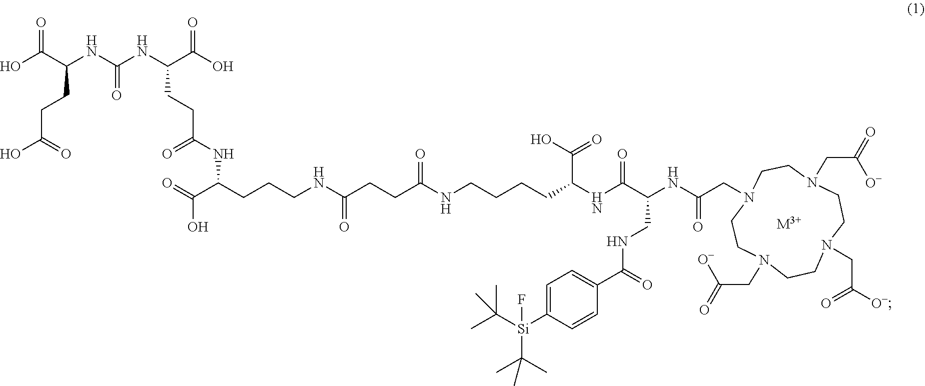
or a pharmaceutically acceptable salt thereof, wherein M³⁺ is a chelated radioactive or non-radioactive metal cation.

2. The compound according to claim 1, wherein M³⁺ is selected from the cations of Sc, Cu, Ga, Y, In, Tb, Ho, Lu, Re, Pb, Bi, Ac, Er and Th. 10 3. The compound according to claim 1, wherein M³⁺ is Lu³⁺.

4. The compound according to claim 1, wherein M³⁺ is ¹⁷⁷Lu³⁺.

5. The compound according to claim 1, wherein M³⁺ is ²²⁵Ac³⁺.

6. The compound according to claim 1, wherein M³⁺ is Ga³⁺.

7. The compound according to claim 1, wherein M³⁺ is ⁶⁸Ga³⁺.

8. The compound according to claim 1 which is: 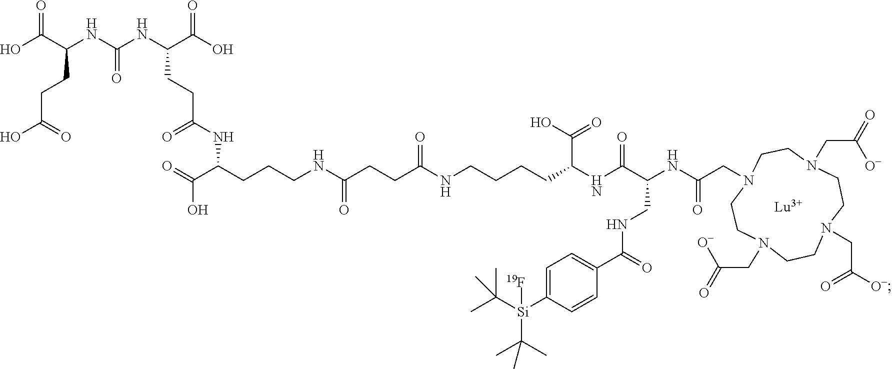
or a pharmaceutically acceptable salt thereof.

9. The compound according to claim 1 which is: 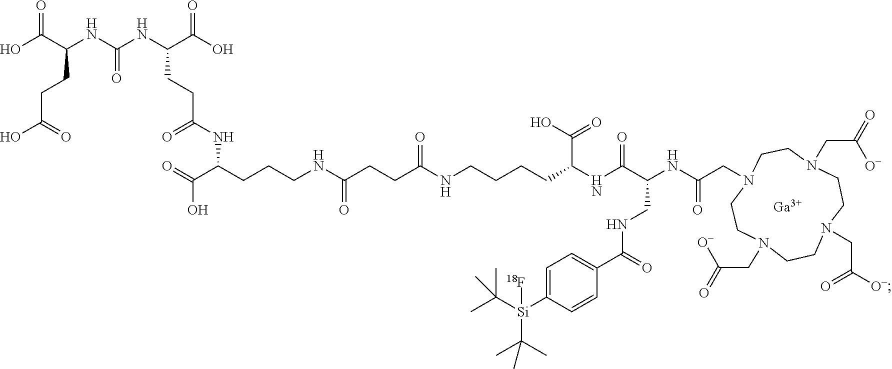
or a pharmaceutically acceptable salt thereof.

10. A pharmaceutical or diagnostic composition comprising one or more compounds according to any one of claims 1 to 9.

11. A compound or composition according to any one of claims 1 to 10 for use as a cancer diagnostic or imaging agent.

12. A compound or composition according to any one of claims 1 to 10 for use in the treatment of cancer.

13. A method of imaging and/or diagnosing cancer comprising administering a compound or composition according to any one of claims 1 to 10 to a patient in need thereof.

14. A compound or composition according to any one of claims 1 to 10 for use as a cancer diagnostic or imaging agent or for use in the treatment of cancer wherein the cancer is prostate, breast, lung, colorectal or renal cell carcinoma.

15. A compound or composition according to any one of claims 1 to 10 for use in the diagnosis, imaging or prevention of neoangiogenesis/angiogenesis.
